(19) United States
(12) Patent Application Publication
(10) Pub. No.: US 2006/0040859 A1
(43) Pub. Date: Feb. 23, 2006

(54) Peptides, DNAs, RNAs, and compounds for inhibiting or inducing adrenomedullin activity, and use of the same

(76) Inventor: Masanobu Kobayashi, Sapporo-shi (JP)

Correspondence Address: FISH & RICHARDSON P.C., PO BOX 1022, MINNEAPOLIS, MN, 55440-1022, US

(21) Appl. No.: 10/508,272

(22) Filed: Sep. 17, 2004

Related U.S. Application Data
(63) Continuation-in-part of application No. PCT/JP03/03344, filed on Mar. 19, 2003.

(30) Foreign Application Priority Data
Mar. 19, 2002 (JP) 2002-075575

(51) Int. Cl. A61K 38/54; C07K 14/575; C07H 21/04

(52) U.S. Cl. 514/12; 530/350; 536/23.5

(57) ABSTRACT

Pharmaceutical compositions containing adrenomedullin antagonist peptides, DNA, RNA compounds inhibitory on adrenomedullin activity, resulting in the efficient blockade of the induction of macroangiogenesis or vasculogenesis of more than 8 μm (in case of murine models), suppress the proliferation of cancer cells due to that inhibitory effect and suppress the protective activity of adrenomedullin. Accordingly, the pharmaceutical compositions of the present invention described herein can be used to treat various cancers, including, but not limited to, stomach cancer, colorectal cancer, lung cancer, ovarian cancer, liver cancer, and pancreatic cancer. Pharmaceutical compositions containing adrenomedullin expression vectors described herein induce macroangiogenesis and, accordingly are effective in treating cardiovascular and renal diseases such as congestive heart failure, myocardial infarction, hypertension, chronic renal failure, stroke, diabetes mellitus, and septic shock.

This application is a continuation-in-part of International Application Serial No. PCT/JP03/03344 filed on Mar. 19, 2003, the content of which are hereby incorporated by reference in its entirety.

### TECHNICAL FIELD

The present invention relates to peptides, composites containing DNA, RNA or compounds inhibitory on adrenomedullin activity, blocking the induction of thick angiogenesis (macroangiogenesis) or vasculogenesis of more than 8 μm (in case of murine models), and having an inhibitory action against cancer vasculatures, vasculogenesis that are effective in treating cancer, and pharmaceutical compositions comprising the same as well as methods of treating cancer using such peptides and pharmaceutical compositions, and to recombinant adrenomedullin expression vectors that are effective in treating cardiovascular and renal diseases such as congestive heart failure, myocardial infarction, hypertension, chronic renal-failure, stroke, diabetes mellitus, and septic shock, and pharmaceutical compositions comprising the same as well as methods of treating cardiovascular diseases through the generation of thick angiogenesis or vasculogenesis of more than 8 μm (in case of murine models) and, thus is effective in treating cardiovascular and renal diseases such as congestive heart failure, myocardial infarction, hypertension, chronic renal failure, stroke, diabetes mellitus, and septic shock using such peptides and pharmaceutical compositions.

### BACKGROUND ART

Recently, cancer has become the second cause of human death following heart disease. Cancer is usually treated by surgical operation, radiotherapy, chemotherapy, immunotherapy, hyperthermia, etc. In all these treatments, the removal of viable cells and induction of cell death are important objectives in order to exterminate cancer cells.

As mentioned above, the goal of cancer treatment is extermination of cancer cells. Gene therapy methods for introducing into cancer cells genes that induce cancer cell death or enhance immune response have been developed and represent viable approaches for treating cancer.

However, regarding gene therapy methods that involve the introduction genes that induce cancer cell death, there is currently no method to introduce genes into all cancer cells. Thus, it is difficult to achieve the intended goal, i.e., the extermination of cancer cells. Furthermore, regarding gene therapy methods involving the introduction of genes that enhance immune response, since the immune response is complicatedly regulated at multiple steps, it has proven to be difficult to enhance the immune response by manipulating one gene alone.

On the other hand, for disorders caused by the depletion of a particular intracellular substance, such as immunodeficiency, gene therapy methods introducing into cells genes that supplement the substance have been shown to be successful. Therefore, such a strategy may also be important in gene therapy for cancer, namely, treating cancer by producing a certain substance.

Adrenomedullin, first discovered in human pheochromocytoma, is a peptide comprising 52 amino acids (Kitamura, K. et al. “Adrenomedullin, a novel hypotensive peptide isolated from human pheochromocytoma”, Biochem. Biophys. Res. Commun. 192: 553-560 (1993)). Adrenomedullin is a hypotensive peptide produced from a preprohormone comprising 185 amino acids through successive enzymatic degradations and amidation. Through these enzymatic degradation and amidation steps, adrenomedullin comprising 52 amino acids is produced.

Adrenomedullin is present in many tissues, including normal adrenal/medulla, atrium, ventricle, endothelial cell, lung, brain, kidney, and bone, and is known to have vasodilating activity. Many roles of AM as vasodepressor were studied (C. Nuki et al., Biochem.Biophys.Res.Commun. 196, 245 (1993); Q. Hao et al., Life Sci. 54, 265(1994); D. Y. Cheng et al., Life Sci., and 55, 251 (1994); C. J. Feng, B. Kang, A. D. Kaye, P. J. Kadowitz, B. D. Nossaman, Life Sci., 433 (1994)). AM activates adenyl cyclase activity through the specific receptor of plasmlemma, and it works so that the flow of calcium2+ to a target cell may be adjusted (270 S. Eguchi et al., Endocrinology 135, 2454 (1994); Y. Shimekake et al., J. Biol. Chem. 4412 (1995)). These signal transduction paths include accommodation of secretion of hormone with regards to many physiological processes. It is corroborated well that accommodation intracellular [cAMP] adjusts bleedoff of the hormone in the pancreas (Y Korman, S. J. Bhathena, N. R. Voyles, H. KOie, L. Recant, Diabetes 34, 717 (1985); C. B. Wollheim, Diabetes 29, 74 (1980)). 3 0 Adrenomedullin is implicated in angiogenesis in reproductive organs, through its vasodilating activity (Oehler M K, Hague S, Rees M C, Bicknell R. Oncogene. Apr. 25, 2002; 21 (18): 2815-21. Adrenomedullin promotes formation of xenografted endometrial tumors by stimulation of autocrine growth and angiogenesis, Zhao Y, Hague S, Manek S, Zhang L, Bicknell R, Rees M C. Oncogene. January 22; 16(3): 409-15(1998). PCR display identifies tamoxifen induction of the novel angiogenic factoradrenomedullin by a non estrogenic mechanism in the human endometrium), but nothing has been known about the role of angiogenesis or vasculognesis in tumor tissues at the time of the present invention. Moreover nothing has been implicated so far on the role of adrenomedullin in generation of thick blood vessels more than 8 μm, namely in macroangiogenesis in any normal and neoplastic tissues including cardiovascular and renal tisuues.

The adrenomedullin content has been shown to be higher in various cancer cells as compared to normal cells and some reported the inhibition of in vitro tumor growth by bocking the function of drenomedullin as an autocrine growth factor of tumor cells (Ouafik L, Sauze S, Boudouresque F, Chinot O, Delfmo C, Fina F; Vuaroqueaux V, Dussert C, Palmari J, Dufour H, Grisoli F, Casellas P, Brunner N, Martin P M. Am J Pathol. April; 160(4): 1279-92(2002). Neutralization of adrenomedullin inhibits the growth of human glioblastoma celllines in vitro and suppresses tumor xenograft growth in vivo). This publication after our invention provided the information that human glioblastoma cell lines express high levels of adrenomedullin mRNA, and that immunoreactive adrenomedullin is released into the culture medium. Real-time quantitative reverse transcriptase-polymerase chain reaction analysis showed that adrenomedullin mRNA was correlated to the tumor type and grade, with high expression in all glioblastomas analyzed, whereas a low expression was found in anaplastic astrocytomas and barely detectable levels in low-grade astrocytomas and oligodendrogliomas. It was shown that exogenously added adrenomedullin can stimulate the growth of these glioblastoma cells in vitro, suggesting that adrenomedullin may function as an autocrine growth factor for glioblastoma cells. A polyclonal antibody specific to adrenomedullin, blocks the binding of the hormone to its cellular receptors and decreases by only 33% the growth of U87 glioblastoma cells in vitro. Intratumoral administration of the anti-adrenomedullin polyclonal antibody resulted in a 70% reduction in subcutaneous U87 xenograft weight. This discrepancy between the in vitro and in vivo effects clearly indicates the presence of so far unknown functions of adrenomedullin in tumor growth in vivo except its nature as an autocrine tumor growth factor.

Prevention or therapy approach of cancer by contacting effective dose of adrenomedullin antibody to adrenomedullin peptide was also reported (WO 97/07214, Published Japanese Translation of International Publication No. Hei 11-512087, leaving a controversial discussion on the role of adrenomedullin or its peptides. They disclosed that an monoclonal antibody against adrenomedullin peptide called PO72 (22-52; corresponiding the sequence SED ID NO: 2 of the present invention) inhibited the in vitro growth of tumor cell lines, NCI-H157, NCI-H720, MCF-7, SNUC1 and NIH:OVCAR-3, clearly indicating the tumor growth promoting activity of adrenomedullinn antagonist peptide AMA.

In addition, several reports demonstrated that the disruption of adrenomedullin induced the growth inhibition of several cancer cell lines (Oehler M K, et al. “Adrenomedullin inhibits hypoxic cell death by upregulation of Bcl-2 in endometrial cancer cells: a possible promotion mechanism for tumour growth”, Oncogene, 20, 2937-2945 (2001); Oehler M K, et al. “Adrenomedullin promotes formation of xenografted endometrial tumors by stimulation of autocrine growth and angiogenesis”, Oncogene, 21, 2815-1821 (2002); Fernandez-Sauze S, et al. “Effects of adrenomedullin on endothelial cells in the multistep process of angiogenesis: involvement of CRLR/RAMP2 and CRLR/RAMP3 receptors”, Int J Cancer, 108, 797-804 (2004)). Even in 2004, the paper described that earlier studies have shown that adrenomedullin, a potent vasodilator peptide, has a variety of cardiovascular effects. However, whether AM has angiogenic potential remains unknown (Tokunaga N, Nagaya N, Shirai M, Tanaka E, Ishibashi-Ueda H, Harada-Shiba M, Kanda M, Ito T, Shimizu W. Tabata Y. Uematsu M, Nishigami K, Sano S, Kangawa K, Mori H. Adrenomedullin gene transfer induces therapeutic angiogenesis in a rabbit model of chronic hind limb ischemia: benefits of a novel nonviral vector, gelatin. Circulation. February 3;109(4):526-31(2004)).

Apart from the controversial findings aforementioned, the inventors discovered the new roles of adrenomedullin and AMA in angiogenesis and vasculogenesis, especially in macroangiogenesis, in tumor tissues and the inventors had finally completed the present creative invention.

Since angiogenesis is necessary for the proliferation of cancer cells surrounded in cancer stromal tissues, cancer cell proliferation can be suppressed by inhibiting angiogenesis (Hanahan D, Folkman J.Cell. August 9;86(3):353-64(1996). Patterns and emerging mechanisms of the angiogenic switch during tumorigenesis). Therefore, a compound capable of inhibiting tumor angiogenesis may prove to be efficacious in treating cancers. Angiogenesis is activated during multistage tumorigenesis prior to the emergence of solid tumors. The treatment with angiogenesis inhibitors can inhibit the progression of tumorigenesis after the switch to the angiogenic phenotype. In general some carcinomas develop from multifocal, hyperproliferative nodules that show the histological hallmarks of human carcinoma in situ. Mice treated with a combination of the angiogenesis inhibitor AGM-1470 (TNP470), the antibiotic minocycline, and interferon alpha/beta markedly attenuated tumor growth but did not prevent tumor formation; tumor volume was reduced to 11% and capillary density to 40% of controls. This study suggest that angiogenesis inhibitors represent a valid component of anticancer strategies aimed at progression from discrete stages of tumorigenesis (Parangi S, O'Reilly M, Christofori G. Holmgren L, Grosfeld J, Folkman J, Hanahan D. Antiangiogenic therapy of transgenic mice impairs de novo tumor growth. Proc Natl Acad Sci U S A. March 5; 93 (5): 2002-7(1996)). So far known antiangiogenic compounds, either endogeneous or exogeneous, only attenuate the capillary density in tumor tissues and this may be one of the reasons of insufficient anticancer effects of so far known antiangiogenic compounds.

Therefore, a compound capable of inhibiting adrenomedullin-induced macroangiogenesis and vasculogenesis, instead of so far well-known angiogenic factors such as VEGF, FGF, HGF et al. playing roles in only microangiogenesis (generation of blood vessels below 8 μm diameter), but not in macroangiogenesis and vasculogenesis, once discovered, may prove to be efficacious in treating various cancers, and a composite capable to produce adrenomedullin efficiently upon in vivo application, once discovered, may also prove to be efficacious in treating a wide range of cardiovascular and renal diseases due to impaired or injured blood vessels such as congestive heart failure, myocardial infarction, hypertension, chronic renal failure, stroke, diabetes mellitus, and septic shock.

Thus, a compound pharmacologically useful by inhibiting adrenomedullin functions either at the level of receptor binding or at the level of intracellular signalling of the ligand to nucleus has not been provided. To the best of the present inventor's knowledge at the time of the completion of the invention, inhibition of adrenomedullin functions had been known to inhibit the proliferation of tumor cells by blocking the function of adrenomedullin as an autocrine tumor growth factor.

Accordingly, an objective of the present invention is to provide novel compounds, particularly andrenomedullin antagonist peptides (AMA), recombinant vectors and the compounds sharing the similar pharmacological functions with AMA, suitable for treating various cancers, including, but not limited to, stomach cancer, colorectal cancer, lung cancer, ovarian cancer, liver cancer, and pancreatic cancer.

The other aim of the present invention is to provide novel compounds, particularly, recombinant andrenomedullin vectors, suitable for treating various cardiovascular and renal diseases such as congestive heart failure, myocardial infarction, hypertension, chronic renal failure, stroke, diabetes mellitus, and septic shock.

### SUMMARY OF THE INVENTION

Noting that adrenomedullin is expressed at high levels in cancer cells, the present inventors considered the use of antagonists of adrenomedullin in cancer treatment and searched for such novel compounds usable in treating cancer.

As a result of exhaustive studies, the present inventors have discovered that a peptide comprising a partially deleted or truncated form of human adrenomedullin or composites containing DNA or RNA with small molecular weights such as a dominant negative adrenomedullin, antisense adrenomedullin, small inhibitory RNA (siRNA) against adrenomedullin or compounds inhibitory on adrenomedullin activity, resulting in the efficient blockade of the induction of thick angiogenesis or vasculogenesis of more than 8 μm (in case of munne models), have an inhibitory action against cancer vasculatures, namely against intratumoral angiogenesis and vasculogenesis and, thus, is effective in treating cancer. This invention also provides the new way to screen new compounds capable to inhibit tumor angiogenic functions of adrenomedullin, to the extent comparable to aforementioned antagonists.

In prarell with the discoveries the present inventors have discovered the effect of intramuscular injection of adrenomedullin expression vectors on cardiovascular diseases through the generation of thick angiogenesis or vasculogenesis of more than 8 μm (in case of murine models) and, thus is effective in treating cardiovascular and renal diseases such as congestive heart failure, myocardial infarction, hypertension, chronic renal failure, stroke, diabetes mellitus, and septic shock.

Accordingly, the present invention provides peptides comprising amino acid sequences wherein at least one amino acid has been deleted from the N-terminal side of mature human adrenomedullin, the amino acid sequence of which is set forth in SEQ ID NO: 1. The full length amino acid sequence for the human adrenomedullin, comprising 185 amino acids, is set forth in SEQ ID NO: 8.

The present invention also provides peptides for treating cancer, comprising amino acid sequences wherein at least one amino acid has been deleted from the N-terminal side of the amino acid sequence set forth in SEQ ID NO: 1.

Furthermore, the present invention provides genes comprising DNA having nucleotide sequences encoding the above-described peptides and peptides for treating cancer (hereinafter simply referred to as “peptides”).

Moreover, the present invention provides recombinant vectors comprising DNA encoding the above-described peptides.

In addition, the present invention provides transformants comprising the aforementioned recombinant vectors.

The present invention further provides methods for producing the above-described peptides, comprising the steps of culturing the transformants, producing and accumulating the peptides, and collecting the peptides.

The present invention also provides pharmaceutical compositions, particularly pharmaceutical compositions for treating cancer, comprising the above-described peptides.

In addition, the present invention provides methods of treating cancer comprising administering a peptide or pharmaceutical composition of the present invention to a subject in need thereof. In one preferred embodiment, the pharmaceutical composition comprises an above-described peptide, alone or in combination with a suitable pharmaceutical carrier. In an alternate preferred embodiment, the pharmaceutical composition contains a DNA encoding an above-described peptide, preferably in the form of a recombinant expression vector.

Moreover, the present invention provides recombinant vectors comprising DNA encoding the adrenomedullin peptides.

In addition, the present invention provides transformants comprising the aforementioned recombinant vectors.

The present invention also provides pharmaceutical compositions, particularly pharmaceutical compositions for treating cardiovascular and renal diseases such as congestive heart failure, myocardial infarction, hypertension, chronic renal failure, stroke, diabetes mellitus, and septic shock, comprising the above-described recombinant vectors comprising DNA encoding the adrenomedullin peptide.

In addition, the present invention provides methods of treating cancer, cardiovascular and renal diseases such as congestive heart failure, myocardial infarction, hypertension, chronic renal failure, stroke, diabetes mellitus, and septic shock, comprising administering a peptide, a recombinant vector, a composite containing DNA or RNA, or a compound inhibitory on adrenomedullin activity, resulting in the efficient blockade of the induction of thick angiogenesis or vasculogenesis of more than 8 μm (in case of murine models), posessing an inhibitory action against cancer vasculatures, namely against intratumoral angiogenesis and vasculogenesis, or a recombinant vector comprising DNA encoding the adrenomedullin peptide, or a pharmaceutical composition of the present invention to a subject in need thereof.

In one preferred embodiment, the pharmaceutical composition comprises an above-described peptide, a composite containing DNA, RNA, or a compound alone or in combination with a suitable pharmaceutical carrier. In an alternate preferred embodiment, the pharmaceutical composition contains a DNA encoding an above-described peptide or adorenomedullin, preferably in the form of a recombinant expression vector.

Other features and advantages of the invention will be apparent from the following detailed description, and from the claims.

### BRIEF DESCRIPTION OF THE DRAWINGS

FIG. 1 depicts the expression of adrenomedullin (AM) mRNA upon cultivating various cell lines under various conditions. FIG. 1A depicts the expression of AM mRNA in various cancer cell lines under normoxic (N) and hypoxic (H) conditions. FIG. 1B depicts the expression of AM mRNA in various pancreatic cancer cell lines under the following conditions: normoxic, normal glucose (N—N); normoxic, glucose-deprived (N-L); hypoxic, normal glucose (H—N); and hypoxic, glucose-deprived (H-L). FIG. 1C depicts the expression of AM mRNA in other cancer cell lines under the following conditions: normoxic, normal glucose (N—N); normoxic, glucose-deprived (N-L); hypoxic, normal glucose (H—N); and hypoxic, glucose-deprived (H-L).

FIG. 2 depicts changes in tumor size due to administration to the tumor of adrenomedullin, an adrenomedullin antagonist peptide of the present invention, or physiological saline (control—V3). Administration of adrenomedullin antagonist resulted in a reduction in tumor volume.

FIG. 3 is a photograph depicting tumors extirpated from mice treated with adrenomedullin or an adrenomedullin antagonist peptide of the present invention. Tumors treated with adrenomedullin antagonist were noticeably smaller than tumors treated with adrenomedullin.

FIG. 4 depicts the results of weighing tumors extirpated from mice treated with adrenomedullin or an adrenomedullin antagonist peptide of the present invention. Tumors treated with adrenomedullin antagonist weighed significantly less than tumors treated with adrenomedullin.

FIG. 5 is a series of photographs showing the results of staining tumor tissues with the anti-CD31 antibody. Tissues treated with adrenomedullin are shown on left; tissues treated with an adrenomedullin antagonist of the present invention are shown on right.

FIG. 6 is a series of photographs showing the results of staining tumor cells with the anti-PCNA antibody. Tissues treated with adrenomedullin are shown on left; tissues treated with an adrenomedullin antagonist of the present invention are shown on right.

FIG. 7 depicts the adrenomedullin antagonist expression vectors utilized in the experiments herein. FIG. 7A depicts the gene insert for the AMA214 expression vector (SEQ ID NO: 5). FIG. 7B depicts the gene insert for the AMA224 expression vector (SEQ ID NO: 6). FIG. 7C depicts the gene insert for the p3XFLAG-CMV-AMA expression vector (SEQ ID NO: 7).

FIG. 8 depicts the effects of AMA on the in vivo growth and angiogenesis of cancer cells. FIG. 8A depicts growth curves for pancreatic cancer cells treated with intra-tumoral injection of an AMA expression vector or control vector. Treatment with AMA expression vector induced tumor regression. FIG. 8B depicts the immunohistochemical findings of tumors treated with an AMA expression vector or control vector. CD-31 positive cells were observed only in cells treated with control vector. FIG. 8C depicts growth curves for pancreatic cancer cells treated with intra-muscular injection of an AMA expression vector or control vector. AMA expression vector induced tumor regression. FIG. 8D depicts the immunohistochemical findings of tumors treated with an AMA expression vector or control vector. CD-31 positive cells were observed only in cells treated with control vector. FIG. 8E depicts growth curves for breast cancer cells treated with intra-muscular injection of various concentrations of an AMA expression vector or a control vector. AMA expression vector induced tumor regression. FIG. 8F depicts the immunohistochemical findings of tumors treated with an AMA expression vector or control vector. As shown in the upper panels, CD-31 positive cells were observed only in cells treated with control vector. As shown in the lower panels, FLAG positive cells were observed in tumors treated with AMA expression vector.

FIG. 9 depicts the in vitro effects of AM and AMA on endothelial cells. FIG. 9A depicts the effects of AM on the growth of HUVEC cells under normoxic conditions. FIG. 9B depicts the effects of AM on the growth of HUVEC cells under hypoxic and nutrient-deprived conditions, in the presence of growth factors VEGF and FGF. FIG. 9C depicts the effects of AMA on the suppression of apoptosis of HUVEC cells induced by AM.

FIG. 10 depicts the schema to screening copmpounds of AM inhibitor by luciferase assay.

FIG. 11 depicts the schematic representation of the seruence of adrenomedullin.

### DETAILED DESCRIPTION OF THE INVENTION

The words “a”, “an”, and “the” as used herein mean “at least one” unless otherwise specifically indicated.

Herein, the present inventors demonstrate that adrenomedullin antagonist (AMA) peptides suppressed the growth of pancreatic cancer cells in vivo when injected intra-tumorally. In addition, injection of AMA expression vectors into the tumor tissues and the femoral muscles significantly reduced the in vivo growth of pancreatic and breast cancer cell lines. Immunohistochemical analyses demonstrate that blood vessel almost completely disappeared in the tumor tissues treated with AMA. Furthermore, endothelial cells underwent apoptosis when exposed to hypoxic and nutrient-deprived conditions, even in the presence of VEGF and FGF. While adrenomedullin (AM) was shown to protect the endothelial cells from apoptosis induced by hypoxic and nutrient-deprived conditions, AMA suppressed this protective activity of AM.

It is of note that AMA did not exhibit any growth inhibitory effect on the cancer cells in vitro. Collectively, all these results suggest that AM is essential for angiogenesis and vasculogenesis in solid tumor tissues, but not as an autocrine/paracrine growth factors, and that injection of AMA, in peptide or vector form, of siRNA, of antagonistic DNA and of compounds inhibiting the tumor angiogenic and vasculogenic actions of adrenomedullin may be effective in the treatment of solid tumors. This feature is distinguished from so far known antiangiogenic factors in the inhibition of macroangiogenesis and vasculogenesis. This feature is distinguished from so far known antiangiogenic factors in the inhibition of macroangiogenesis and vasculogenesis. Compounds useful to inhibit the tumor angiogenic and vasculogenic actions of adrenomedullin, effective in the treatment of solid tumors are easily screened by the method described below and example. Non-peptidic compounds that regulate adrenomedullin angiogenic functions are so far not known. The method disclosed in the present invention provides an efficient way to screen a large library of small molecules and will be useful to the identification of positive and negative modulators of AM function.

Accordingly, the present invention provides peptide antagonists of adrenomedullin. As used herein, the term “antagonist” refers to a chemical that acts within the body to reduce the physiological activity of another chemical substance, particularly one that opposes the action of a substance occurring naturally in the body by combining with and blocking its receptor or inhibiting the siganlling cascade after a ligand engagement from cell membrane to nucleus. In the context of the present invention, an adrenomedullin antagonist (AMA) is a peptide that inhibits the biological activity of adrenomedullin. In this context, the term “inhibit” encompasses not only suppression of activity, action or function but also reductions in such.

Adrenomedullin (AM), a potent vasodilator have functions ranging from reproduction to blood pressure regulation, namely AM has multi-functional properties, of which the vasodilatory hypotensive effect is the most characteristic. AM and its gene are ubiquitous in the cardiovascular system, as well as the adrenal medulla. AM secretion, especially in cardiovascular tissues, is regulated mainly by mechanical stressors such as shear stress, inflammatory cytokines such as interleukin-1, tumor necrosis factor, and lipopolysaccharide, hormones such as angiotensin II and endothelin-1, and metabolic factors such as hypoxia, ischemia, or hyperglycemia. Elevation of plasma AM due to overproduction in pathological conditions may explain the raised plasma AM levels present in cardiovascular and renal diseases such as congestive heart failure, myocardial infarction, hypertension, chronic renal failure, stroke, diabetes mellitus, and septic shock. In addition to shear stress, stretching of cardiomyocytes may be another mechanical stimulus for AM synthesis and secretion. Nevertheless, almost none was known so far about the effect of AM on the regeneration of blood vessels.

Herein, the present inventors demonstrate that non-viral recombinant vectors containing DNA coding for adrenomedullin peptides re-generated the blood vessels in vivo when injected intra-tumorally. In addition, injection of the expression vectors into the femoral muscles significantly increased the number of blood vessels with diameters over 8 μm, detected by immunohistochemical analyses in the hind limb of rabbits injected with the expression vectors. It is of note that this regeneration of thick blood vessels, namely macroangiogenesis has not been reported for VEGF and FGF.

Hereinafter, the present invention is described in detail.

Peptides:

Peptides of the present invention comprise an amino acid sequence in which at least one amino acid has been deleted from the N-terminal side of a peptide consisting of the amino acid sequence set forth in SEQ ID NO: 1. Specifically, peptides of the present invention comprise amino acid sequences in which 1 to 25, 1 to 20, 1 to 15, 1 to 10, or 1 to 5 amino acids have been deleted from the N-terminal side of a peptide consisting of the amino acid sequence set forth in SEQ ID NO: 1. Herein, the peptide consisting of the amino acid sequence set forth in SEQ ID NO: 1 is adrenomedullin of human origin.

Peptides of the present invention can be obtained by cleaving the peptide consisting of the amino acid sequence set forth in SEQ ID NO: 1 (i.e., human adrenomedullin) with proteolytic enzymes using methods known in the art.

The nucleotide sequence of human adrenomedullin cDNA has been determined (Kitamura, et al., Biochem. Biophys. Res. Commun. 194, 720 (1993)). Furthermore, the amino acids sequence of the 52 amino acid peptide of human adrenomedullin is set forth herein as SEQ ID NO: 1. Therefore, a desired peptide of the present invention can be obtained by synthesizing DNA comprising the nucleotide sequence encoding the desired peptide, incorporating the synthesized DNA into a vector, transforming a host with the vector comprising the DNA, and culturing the transformant thus obtained to produce the peptide. Alternatively, peptides of the present invention can be produced by any peptide synthesis method conventionally known in the art.

Peptides of the present invention may also be those in which 1 to 10, preferably 1 to 5, amino acids in the C-terminal side have been deleted. Such peptides, having amino acid deletion in the C-terminal side, can be prepared by the peptide synthesis method or transformant method as described above.

Peptides of the present invention can be used as compositions for treating cancer. In addition to the peptides described above, the present invention encompasses peptides that are substantially identical and/or functionally equivalent thereto. Accordingly, the present invention contemplates certain mutations or variants of the disclosed sequences. In general, peptides are considered substantially identical even when 1 to 5, more preferably 1 to 3, amino acids of the whole amino acid sequence of a peptide are deleted, substituted and/or added. Likewise, peptides are considered to be “functionally equivalent” when a subject peptide retains a biologically significant activity that is characteristic of a peptide of the present invention. Examples of biologically significant activities of the peptides of the present invention, such as the peptide of SEQ ID NO: 2, include the ability to oppose the action of adrenomedullin, inhibit macroangiogenesis and suppress the proliferation of cancer cells. Accordingly, the present invention includes peptides of the present invention in which one or more amino acids are substituted, deleted and/or added, so long as the resulting peptide retains the adrenomedullin antagonist activity, able to inhibit angiogenesis and suppress proliferation in cancer cells, and thereby exert a therapeutic effect on cancer. These activities can be routinely assayed for methods well known to those skilled in the art, including those methods discussed herein, particularly in the Examples section. Other amino acids may be deleted, substituted, or added, so long as the resulting peptide is an adrenomedullin antagonist that exerts a therapeutic effect on cancer.

A peptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the peptide of SEQ ID NO: 2, it is within the scope of the present invention.

Methods for preparing peptides functionally equivalent to a given peptide are well known to those skilled in the art and include conventional methods of introducing mutations into a peptide. For example, one skilled in the art can prepare peptides functionally equivalent to a peptide of the present invention (e.g., a peptide of SEQ ID NO: 2) by introducing appropriate deletions, substitutions, insertions and/or additions into the amino acid sequence of either of these proteins by site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-9456 (1984); Kramer and Fritz, Methods Enzymol 154: 350-67 (1987); Kunkel, Proc Natl Acad Sci USA 82: 488-92 (1985); Kunkel, Methods Enzymol 85: 2763-6 (1988)). Mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Amino acid mutations can occur in nature, too. Accordingly, the present invention includes peptides having amino acid sequence set forth in SEQ ID NO: 1 in which at least one amino acid has been deleted from the N-terminal side of a peptide, further wherein one or more amino acids are deleted, substituted and/or added, provided the resulting mutated peptide is functionally equivalent to the peptide of SED ID NO: 2.

The number of amino acids that may be deleted, substituted and/or added is not particularly restricted, so long as biologically significant activity is maintained. Generally, up to about 50 amino acids may be deleted, substituted and/or added, preferably up to about 30 amino acids, more preferably up to about 10 amino acids, and even more preferably up to about 3 to 5 amino acids. Likewise, the site of mutation is not particularly restricted, so long as the deletion, substitution, insertion and/or addition does not result in the disruption of biologically significant activity.

Amino acid substitutions may be made at one or more predicted, preferably nonessential amino acid residues. A “nonessential” amino acid residue is a residue that can be altered from the wild-type sequence of a protein (e.g., the sequences shown in SEQ ID NO: 2) without altering the biological activity, whereas an “essential” amino acid residue is required for biological activity. The amino acid residue to be mutated is preferably substituted for a different amino acid that allows the properties of the amino acid side-chain to be conserved (a process known as conservative amino acid substitution). Herein, the phrase “conservative amino acid substitution” refers to the replacement of an amino acid residue replaced with an amino acid residue having a chemically similar side chain. Groups of amino acid residues having similar side chains have been defined in the art. Examples of amino acid groups include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Additional examples of amino acid groupings include side chains having the following chains properties, characteristics, and/or functional groups or characteristics in common: hydrophobic amino acids (A, I, L, M, F, P, W, Y, V), hydrophilic amino acids (R, D, N, C, E, Q, G, H, K, S, T), aliphatic side-chains (G, A, V, L, I, P); a hydroxyl containing side-chains (S, T, Y); sulfur atom containing side-chains (C, M); carboxylic acid and amide containing side-chains (D, N, E, Q); base containing side-chains (R, K, H); and aromatic containing side-chains (H, F, Y, W). Note, the parenthetic letters indicate the one-letter codes of amino acids.

An example of a peptide in which one or more amino acids residues are added is a fusion protein containing a peptide of the present invention. Fusion proteins are fusions of a peptide of the present invention with other peptides or proteins, and are included in the present invention. Fusion proteins can be made by techniques well known to a person skilled in the art, such as by linking the DNA encoding a peptide of the present invention with DNA encoding other peptides or proteins, so that the frames match, inserting the fusion DNA into an expression vector and expressing it in a host. There is no restriction as to the peptides or proteins fused to the peptide of the present invention.

Known peptides that can be used as peptides that are fused to a peptide of the present invention include, for example, FLAG (Hopp et al., Biotechnology 6: 1204-10 (1988)), 6×His containing six His (listidine) residues, 10×His, Influenza agglutinin (HA), human c-myc fragment, VSP-GP fragment, p18HIV fragment, T7-tag, HSV-tag, E-tag, SV40T antigen fragment, lck tag, α-tubulin fragment, B-tag, Protein C fragment, and the like. Examples of proteins that may be fused to a peptide of the invention include GST (glutathione-S-transferase), Influenza agglutinin (HA), immunoglobulin constant region, β-galactosidase, MBP (maltose-binding protein), and such.

Fusion proteins can be prepared by fusing commercially available DNA, encoding the fusion peptides or proteins discussed above, with the DNA encoding a peptide of the present invention and expressing the fused DNA prepared. The C-terminus of a peptide of the present invention is usually a carboxyl group (—COOH) or carboxylate (—COO⁻), but may also be an amide (—CONH₂) or ester (—COOR). Herein, examples of R in esters include C₁₋₆ alkyl groups such as methyl, ethyl, n-propyl, isopropyl, and n-butyl; C₃₋₈ cycloalkyl groups such as cyclopentyl and cyclohexyl; C₆₋₁₂ aryl groups such as phenyl and α-naphthyl; and pivaloyloxymethyl ester, which is widely used as an oral ester.

In addition, when the above-described peptides have a carboxyl group (or carboxylate) at positions other than the C-terminus, such carboxyl groups may be amidated or esterified. For example, esters, such as the aforementioned C-terminal esters, may be used as peptides of the present invention. Furthermore, the present invention also include peptides whose glutamic acid residue at the N-terminus generated by an in vivo cleavage is converted into pyroglutamic acid; peptides in which OH, COOH, NH₂, SH, and such on the side chains of intramolecular amino acids are protected with appropriate protecting groups (e.g., C₁₋₆ acyl groups such as formyl group and acetyl group); and conjugated proteins, such as glycoproteins, which are bound with sugar chains.

Salts of the above-described peptides are preferably those of physiologically acceptable acids in particular. Such salts include those with inorganic acids (e.g., hydrochloric acid, phosphoric acid, hydrobromic acid, and sulfuric acid) and with organic acids (e.g., acetic acid, formic acid, propionic acid, fumaric acid, maleic acid, succinic acid, tartaric acid, citric acid, malic acid, oxalic acid, benzoic acid, methanesulfonic acid, and benzenesulfonic acid).

Peptides for Treating Cancer:

Peptides for treating cancer of the present invention are described below.

Peptides for treating cancer of the present invention are those having the amino acid sequence wherein at least one amino acid has been deleted from the N-terminal side of a peptide consisting of the amino acid sequence set forth in SEQ ID NO: 1. Specifically, the peptides of the present invention have the amino acid sequence where 1 to 25, 1 to 20, 1 to 15, 1 to 10, or 1 to 5 amino acids have been deleted from the N-terminal side of a peptide consisting of the amino acid sequence set forth in SEQ ID NO: 1. Herein, the peptide consisting of the amino acid sequence set forth in SEQ ID NO: 1 is adrenomedullin of human origin.

Peptides for treating cancer of the present invention can be obtained by cleaving the peptide consisting of the amino acid sequence set forth in SEQ ID NO: 1 with a proteolytic enzyme using methods known in the art.

The nucleotide sequence of the human adrenomedullin cDNA has been determined (Kitamura, et al. Biochem. Biophys. Res. Commun. 194, 720 (1993)). Therefore, a desired peptide for treating cancer can be obtained by synthesizing DNA comprising the nucleotide sequence encoding the desired peptide, incorporating this synthesized DNA into a vector, transforming a host with the vector comprising the DNA, and culturing the transformant thus obtained to produce the peptide. The peptide can also be produced using conventional peptide synthesis methods known in the art.

Peptides for treating cancer of the present invention may also be those in which 1 to 10, preferably 1 to 5, amino acids in the C-terminal side have been deleted. Such peptides for treating cancer, having amino acid deletion in the C-terminal side, can be prepared by the peptide synthesis method or the transformant method as described above.

In peptides for treating cancer of the present invention, similarly as in the above-described peptides of the present invention, other amino acids may be deleted, substituted, or added.

Further, the amides, esters, or salts of the peptides for treating cancer are similar to those of the above-described peptides of the present invention.

A specific example of a peptide and peptide for treating cancer of the present invention is the peptide comprising the amino acid sequence set forth in SEQ ID NO: 2, ie., a 31 amino acid peptide comprising a truncated form of human adrenomedullin in which the first 21 amino acids of the N-terminal of human adrenomedullin have been cleaved.

Peptides and peptides for treating cancer of the present invention can be used in the treatment of various cancers, including, but not limited to, stomach cancer, colorectal cancer, lung cancer, ovarian cancer, liver cancer, and pancreatic cancer. When introduced into cancer cells, the peptides and peptides for treating cancer of the present invention are believed to inhibit angiogenesis of cancer cells and suppress the proliferation of cancer cells through such an inhibitory effect.

Anti-cancer activities of the peptides and peptides for treating cancer of the present invention can be assessed, for example, by administering these peptides to animals, such as mice, in which cancer has been generated, and investigating the disappearance of cancer cells.

Nucleotides, Vectors, and Host Cells:

Next, genes having DNA comprising the nucleotide sequence encoding the peptides or peptides for treating cancer of the present invention (hereinafter also simply referred to as “peptides”) are described. As described above, the cDNA of the human adrenomedullin has been sequenced. Therefore, based on the published nucleotide sequence, a desired peptide can be obtained by synthesizing a DNA molecule comprising the nucleotide sequence encoding the desired peptide.

A recombinant vector comprising DNA encoding a peptide of the present invention can be prepared according to conventional methods known in the art, for example, by ligating (inserting) a nucleotide sequence encoding a peptide of the present invention to an appropriate vector. The present invention is not limited to a particular vector, so long as the vector can replicate in a selected host. Examples include plasmid DNA and phage DNA.

A recombinant vector can be prepared by excising a DNA fragment comprising DNA encoding a peptide of the present invention, and ligating it downstream of a promoter within an appropriate expression vector. Suitable vectors for use in the present invention include plasmids derived from Escherichia coli (e.g., pBR322, pBR325, pUC18, pUC19, pUC118, or pBluescript); plasmids derived from Bacillus subtilis (e.g., pUB110, pTP5, or pC194); plasmids derived from yeast (e.g., pSH19, pSH15, YEp13, or YCp50); bacteriophages, such as λ phage; and animal viruses, including, but not limited to, retrovirus, vaccinia virus, or baculovirus. In the context of the present invention, any promoters may be used so long as they are adapted for use in hosts used to express the gene of interest. For example, preferred promoters are as follows: when the host is E. coli, the trp promoter, lac promoter, recA promoter, λPL promoter, 1 pp promoter, T7 promoter, T3 promoter, and araBAD promoter; when the host belongs to the genus Bacillus, the SPO1 promoter, penP promoter, XYL promoter, HWP promoter, and CWP promoter; when the host is Bacillus subtilis, the SPO1 promoter, SPO2 promoter, and penP promoter; and when the host is yeast, the PHO5 promoter, PGK promoter, GAP promoter, and ADH promoter. When animal cells are used as the host, usable promoters include the SRα promoter, SV40 promoter, LTR promoter, CMV promoter, HSV-TK promoter, etc. In addition, when insect cells are used as the host, the polyhedron promoter, OplE2 promoter, and such are preferred.

In addition to the above described promoter, the expression vector may include, if desired, an enhancer, splicing signal, poly(A) addition signal, selection marker, SV40 replication origin (abbreviated to “SV40ori” sometimes hereinafter), and such, which are known in the art. Furthermore, if needed, the protein encoded by a DNA of the present invention can also be expressed as a fusion protein with another protein (e.g., glutathione-S-transferase and protein A). Such fusion proteins can be cleaved using a site-specific protease to divide them into respective proteins.

Examples of suitable host cells include Escherichia bacteria, Bacillus bacteria, yeast, insect cells, insects, and animal cells. Specific examples of suitable Escherichia bacteria include Escherichia coli K12-DH1 (Proc. Natl. Acad. Sci. USA 60, 160 (1968)), JM103 (Nucleic Acids Research 9, 309 (1981)), JA221 (Journal of Molecular Biology 120, 517 (1978)), HB101 (Journal of Molecular Biology 41, 459 (1969)), DH5α, and JM109. Examples of suitable Bacillus bacteria include Bacillus subtilis MI114 (Gene 24, 255 (1983)), 207-21 (Journal of Biochemistry 95, 87 (1984)), and Bacillus brevis. Examples of suitable yeasts include Saccaromyces cerevisiae AH22, AH22R-, NA87-11A, DKD-5D, 20B-12, Schizosaccaromyces pombe NCYC1913, NCYC2036, Pichia pastoris, and Hansenula polymorpha. Examples of suitable animal cells include simian COS-7 cells, Vero cells, Chinese hamster ovary cells (hereinafter abbreviated to CHO), Chinese hamster ovary cells deficient in dhfr gene (hereinafter abbreviated to CHO(dhfr⁻), mouse L cells, mouse AtT-20 cells, mouse myeloma cells, rat GH3 cells, human FL cells, and HEK293 cells.

Transformation of the above-described host cells can be carried out according to methods known in the art. Exemplary methods for transforming host cells are described in the following references: Proc. Natl. Acad. Sci. USA 69, 2110 (1972); Gene 17, 107 (1982); Molecular & General Genetics 168, 111 (1979); Methods in Enzymology 194, 182-187 (1991); Proc. Natl.

Acad. Sci. USA 75, 1929 (1978); Cell Technology (Suppl. 8) New Cell Technology Experimental Protocol 263-267 (1995) (Shujunsha); and Virology 52, 456 (1973).

When a plant is transformed with a gene encoding a peptide of the present invention to obtain a transgenic plant, the gene can be introduced into the plant, for example, by the electroporation method, the Agrobacterium method, the particle gun method, or the PEG method.

For example, when the electroporation method is used, the gene is introduced into the host in an electroporation device equipped with a pulse controller, under the conditions of 500 to 600 V and 100 μF for 20 msec.

When the Agrobacterium method is used, a transgenic plant can be obtained by introducing a plant expression vector construct into an appropriate Agrobacterium, such as Agrobacterium tumefaciens, and infecting a leaf fragment of a host with such a strain under aseptic conditions according to, for example, the vacuum infiltration method (described in Bechtold et al., C. R. Acad. Sci. Ser. III Sci. Vie, 316, 1194-1199 (1993)).

When the particle gun method is employed, plant bodies, plant organs (e.g., leafs, petals, stems, roots, and seeds), and plant tissues (e.g., epidermis, phloem, parenchyma, xylem, and vascular bundle) are used as is, or sections or protoplasts may be prepared therefrom for use.

Samples thus prepared are then treated using a gene transfer apparatus (e.g., BIOLISTIC POS 1000/He, BioRad). The treatment is usually performed under a pressure of about 1000 to 1100 psi and a distance of around 5 to 10 cm; however, treatment conditions may vary depending on plants and samples.

Further, plants usable in transformation may be any of conifers, broad-leaved trees, dicotyledons, monocotyledons, etc.

Methods for introducing recombinant vectors into bacteria such as E. coli are not particularly limited, so long as they successfully introduce DNA into the selected bacteria. For example, the calcium ion method (Cohen, S. N. et al., Proc. Natl. Acad. Sci. USA 69, 2110 (1972)) and the electroporation method may be used.

When yeast is used as the host, the methods for introducing recombinant vectors therein are not particularly limited, so long as they allow for successful introduction of DNA into yeast. For example, the electroporation method, the spheroplast method, and the lithium acetate method may be used.

When animal cells are used as the host, the methods for introducing recombinant vectors therein are not particularly limited, so long as they allow for successful introduction of DNA into the animal cells. For example, the electroporation method, the calcium phosphate method, and the lipofection method may be used.

When insect cells are used as the host, methods for introducing recombinant vectors into insect cells are not particularly limited, so long as they allow for successful introduction of DNA into the insect cells. For example, the calcium phosphate method, the lipofection method, and the electroporation method may be used.

Incorporation of a gene into a host can be confirmed, for example, by the PCR method, the Southern hybridization method, and the Northern hybridization method. For example, DNA is prepared from the transformant and DNA-specific primers are designed to perform PCR. PCR is carried out under conditions similar to those used in preparing the above-described plasmids. Then, amplified products are subjected to, for example, agarose gel electrophoresis, polyacrylamide gel electrophoresis, or capillary electrophoresis, and stained with ethidium bromide, SYBR Green solution, etc. The amplified product detected as a single band indicates successful transformation. Amplified products may also be detected by performing PCR using primers labeled with fluorescent dye and such in advance. Furthermore, amplified products can be identified through fluorescence or enzymatic reaction after fixing them on a solid phase, such as a microplate.

A peptide of the present invention can be prepared by culturing an aforementioned transformant, producing and accumulating the peptide, and collecting the peptide. In the context of the present invention, the peptide may be accumulated through the culture not only in culture supernatants but also in any of cultured cells, cultured bacterial cells, or the lysate of cells or bacteria. In the present invention, methods for culturing transformants are not particularly limited, and any method conventionally used in culturing hosts may be used.

For example, when the host is a microorganism, such as E. coli or yeast, the culture medium may be either a natural medium or a synthetic medium, so long as it contains the carbon source, nitrogen source, minerals, and such, to be metabolized by the microorganism, and is capable of efficiently culturing the transformant. Examples of carbon sources include carbohydrates, such as glucose, fructose, sucrose, and starch; organic acids, such as acetic acid and propionic acid; and alcohols, such as ethanol and propanol. Examples of nitrogen sources include ammonia; ammonium salts of inorganic or organic acids, such as ammonium chloride, ammonium sulfate, ammonium acetate, and ammonium phosphate; other nitrogen-containing compounds; and peptone, meat extracts, and corn steep liquor. Examples of mineral sources include potassium dihydrogenphosphate, dipotassium hydrogenphosphate, magnesium phosphate, magnesium sulfate, sodium chloride, ferrous sulfate, manganese sulfate, copper sulfate, and calcium carbonate. The culture is usually performed under aerobic conditions by the shake culture, aerated spinner culture, etc. When the host is E. coli, the culture is carried out, at about 15 to 43° C. for about 12 to 48 h. When the host is a Bacillus bacterium, the culture is carried out at about 30 to 40° C. for about 12 to 100 h. When the host is yeast, the culture is performed at about 20 to 35° C. for about 24 to 100 h. If necessary, aeration and stirring may be applied to the culture. When pH adjustment is required, it is typically performed using inorganic or organic acid, alkaline solution, etc.

When using an inducible promoter as the promoter in the recombinant expression vector, the culture medium for the transformants comprising the expression vector may include the addition of an inducer if necessary. For example, when the expression vector of interest includes the T7 promoter, culture may be performed with the addition of IPTG and such to the medium. Furthermore, when the expression vector of interest includes the trp promoter, a promoter inducible with indoleacetic acid (IAA), IAA and such may be added to the medium.

When culturing transformants obtained using animal cells as the host, suitable media include the generally used RPMI1640 medium, DMEM medium, or these media supplemented with fetal bovine serum and such. The culture is usually performed in the presence of about 5% carbon dioxide at about 37° C. for 1 to 30 days.

When a peptide of the present invention is produced through cell culture, a crude extract of the protein may be obtained, for example, by a method comprising: collecting the cells by methods known in the art; suspending them in an appropriate buffer solution; crushing the cells by, for example, sonication, lysozyme, and/or freeze-thawing; and then centrifuging and/or filtering. The buffer solution may contain protein denaturants, such as urea and guanidine hydrochloride, and surfactants, such as Triton X-100®. When the peptide is secreted into the culture medium, the supernatant is collected after the culture and separated from the cells using methods known in the art. The proteins contained in the supernatant or extract thus obtained can be purified by appropriate combinations of separation/purification methods known in the art. That is, the protein of interest can be purified using, for example, ammonium sulfate precipitation, gel chromatography, ion exchange chromatography, and affinity chromatography, either alone or in appropriate combinations.

A peptide of the present invention thus obtained can be converted into a salt by methods known in the art. Similarly, when the peptide is obtained as a salt, it can be converted to the free form or another salt form using methods known in the art. Furthermore, proteins produced by transformants can optionally be fragmented, before or after purification, by treatment with appropriate protein modifying enzymes, such as trypsin and chymotrypsin. In addition, proteins can optionally be modified by treatment with protein modifying enzymes such as kinases. The presence in a sample of a protein of the present invention or a salt thereof can be determined using various binding assays, enzyme immunoassays with specific antibodies, etc.

Pharmaceutical Compositions:

Pharmaceutical compositions and pharmaceutical compositions for treating cancer of the present invention are explained below and are hereinafter also simply referred to as “pharmaceutical compositions”. Pharmaceutical compositions of the present invention contain a peptide of the present invention. The pharmaceutical compositions of the present invention contain pharmaceutically acceptable carriers in addition to a peptide of the present invention. The content of the peptide of the present invention is defined as an amount capable of suppressing cancer cell growth, reducing cancer cells, or exhibiting therapeutic effect on treated patients, when administered to a patient in need thereof. Dosages to be administered to a patient are generally determined based on the body surface area, body weight, symptom, etc. of the patient. The mutual relationship of dosages between animals and humans is known in the art, and the body surface area of a patient can be determined from the height and body weight of patients.

Suitable dosages of peptides of the present invention may range from about 0.1 mg/kg to about 0.5 mg/kg. Dosages are preferably altered according to the administration method and amount of excipient(s); dosages are further altered when other treatment methods, such as other anticancer drugs and radiotherapy, are combined with the peptides.

Pharmaceutical compositions of the present invention can be administered parenterally, for example, subcutaneously, intraperitoneally, intramuscularly, or intravenously. An embodiment of a pharmaceutical preparation for parenteral administration may include an aqueous solution containing about 5% glucose or other pharmaceutically usable excipients and activators known in the art for use in an isotonic salt solution. Solubilizers, such as cyclodextrin and other solubilizers known in the art, are examples of excipients that may be added.

Pharmaceutical compositions of the present invention can be incorporated into a pharmaceutical preparation for administration by other methods known in the art using conventional techniques. For example, the pharmaceutical compositions can be incorporated into pharmaceutical preparations for oral administration, such as capsules, gels, or tablets. Capsules are composed of pharmaceutically acceptable materials known in the art, such as gelatin or cellulose derivatives. Tablets can be obtained by compressing mixtures of peptides of the present invention and solid carriers as well as lubricants, using methods known in the art. Examples of suitable solid carriers include starch and sugar bentonite. The peptides of the present invention can be administered, for example, in the form of hard shell tablets containing lactose or mannitol as binders, known filling materials, and/or tablet-forming reagents, or in the form of capsules.

Pharmaceutical compositions comprising peptides of the present invention can be used to treat various cancers, including, but not limited to, stomach cancer, colorectal cancer, lung cancer, ovarian cancer, liver cancer, and pancreatic cancer. When introduced into cancer cells, the peptides contained in pharmaceutical compositions of the present invention are believed to inhibit angiogenesis of cancer cells, and suppress the proliferation of cancer cells due to that inhibitory effect.

The anti-cancer activity of a pharmaceutical composition of the present invention can be assessed by generating cancer in animals such as mice, then administering to the animals the pharmaceutical composition and examining the disappearance of cancer.

In an alternate embodiment, pharmaceutical compositions of the present invention may contain genes comprising DNA encoding peptides of the present invention,

Pharmaceutical compositions of the present invention can be administered in a dosage form using either non-viral vector or viral vectors.

When using a non-viral vector, DNA encoding a peptide of the present invention can be introduced into cells or tissues using a recombinant expression vector prepared by incorporating the DNA into a commonly used gene expression vector employing techniques as described below. Examples of methods for introducing genes into cells include the calcium phosphate co-precipitation method and the DNA direct injection method using glass micropipette.

Additional examples of methods for introducing genes into tissues include gene transfer methods using internal type liposome, electrostatic type liposomes, HVJ-liposomes, and improved HVJ-liposomes (HVJ-AVE liposome); gene transfer methods mediated by receptors; methods for transferring DNA molecules together with carriers (metal particles) into cells using a particle gun; methods for directly transferring naked-DNA; and transfer methods mediated by positively charged polymers. Herein, suitable expression vectors include pCAGGS (Gene 108, 193-200(1991)), pBK-CMV, pcDNA3.1, and pZeoSV (Invitrogen, Stratagene).

Viral vectors suitable for administration are exemplified by recombinant adenoviruses and retroviruses. Specifically, genes can be introduced into cells using recombinant expression vectors comprising DNA encoding a peptide of the present invention incorporated into DNA viruses or RNA viruses, such as retrovirus, adenovirus, adeno-associated virus, herpes virus, vaccinia virus, poxvirus, poliovirus, Sindbis virus, Sendai virus, SV40, and human immunodeficiency virus (HIV), which have been made a virulent, to infect cells with these recombinant viruses.

Among the aforementioned viral vectors, the infection efficiency of adenovirus is known to be extremely high as compared to other viral vectors. From this point of view, the adenoviral vector system is preferably used.

Pharmaceutical compositions of the present invention containing DNA or RNA also involve antagonistic DNA or small interfering RNA (siRNA), capable to inhibit the transalation of adrenomedullin. The preparation of these composite containing DNA or RNA can be carried out according to methods known in the art.

Pharmaceutical compositions of the present invention can be introduced into patients by the in vivo method for directly introducing pharmaceutical compositions into the body of a patient, or by the ex vivo method for introducing pharmaceutical compositions into a certain type of cells extracted from a patient and then returning the cells back into the body of the patient.

When pharmaceutical compositions of the present invention are administered by the in vivo method, they can be administered through an appropriate route according to the target to be treated, i.e., cells, tissues, and target organs. For example, they may be administered intravenously, intra-arterially, subcutaneously, intracutaneously, intramuscularly, or directly into tissues where the target cancer lesions are localized.

Various pharmaceutical preparation forms (e.g., liquid) are suitable for the above-described administration forms and can be routinely adopted. For example, in the case of an injection comprising DNA as an effective ingredient, the injection can be prepared according to conventional methods by dissolving DNA in an appropriate solvent (e.g., a buffer solution such as PBS, physiological saline, and sterilized water), sterilizing the solution by filtration with a filter and such, if necessary, and then filling the solution into aseptic containers. Commonly used carriers and such may be added to the injection if necessary. Furthermore, liposomes, such as the HVJ-liposome, can be in the form of a liposome preparation, such as a suspension, frozen form, or centrifugation-concentrated frozen form.

Further, in order to facilitate the localization of the gene into the vicinity of the affected region, a sustained release preparation (e.g., mini-pellet) can be prepared to be embedded near the affected region. It is also possible to continuously and slowly administer the gene to the affected area using an osmotic pump or the like.

Selective introduction into cancer cells can be achieved by targeting a cancer antigen specifically expressed on the surface of the cancer cell or a cancer antigen expressed particularly at higher levels in the cancer cell than normal cells (e.g., transferrin receptor and EGF receptor).

For example, pharmaceutical compositions of the present invention can be specifically introduced into cancer cells using immuno-liposomes obtained by enclosing a pharmaceutical composition comprising DNA encoding a peptide of the present invention in liposomes coupled with a monoclonal antibody against a specific surface antigen of cancer cells.

Contents of DNA in the pharmaceutical preparations can be appropriately adjusted according to the disease being treated, the age and body weight of the patient, etc.

Pharmaceutical compositions containing genes comprising DNA encoding peptides of the present invention can be used to treat various cancers, including, but not limited to, stomach cancer, colorectal cancer, lung cancer, ovarian cancer, liver cancer, and pancreatic cancer. Following introduction into cells, the pharmaceutical compositions of the present invention are believed to produce a peptide of the present invention that inhibits angiogenesis of cancer cells and suppresses the proliferation of cancer cells due to that inhibitory effect.

The anti-cancer activity of a particular peptide or pharmaceutical composition of the present invention can be assessed, for example, by generating cancer in animals, such as mice, administering the pharmaceutical composition, and examining changes in the size of cancer cells.

Methods of Treating Cancer:

Peptides and pharmaceutical compositions of the present invention can be used to treat cancer as described above. Accordingly, the present invention provides methods of treating cancer, comprising administering a peptide or pharmaceutical composition of the present invention to a subject in need thereof.

The method of treating cancer of the present invention contemplates both local and systemic administration of either a peptide of the present invention or a pharmaceutical composition containing such a peptide or a DNA encoding such a peptide. As discussed above, suitable administration routes and dosages are known in the art and may be routinely selected by those skilled in the art depending upon the particular circumstances of the subject to be treated. When the pharmaceutical composition to be administered takes the form of an expression vector carrying a DNA encoding a peptide of the present invention, particularly preferred administration methods include, but are not limited to, intratumor and intramuscular injection.

As noted above, the peptides of the present invention inhibit angiogenesis of cancer cells and suppress the proliferation of cancer cells due to that inhibitory effect. Furthermore, as demonstrated in the Examples below, administration of a peptide of the present invention to a cancerous tumor significantly reduced the growth of the tumor, reduced the presence of blood vessels in the tumor tissues, and suppressed the protective activity of adrenomedullin therein. Accordingly, the method of treating cancer of the present invention can be applied to various cancers, including, but not limited to, stomach cancer, colorectal cancer, lung cancer, ovarian cancer, liver cancer, and pancreatic cancer.

Animals to be treated with the peptides or pharmaceutical compositions of the present invention are not particularly limited, and include humans and other mammals, such as, rats, monkeys, dogs, cats, mice, guinea pigs, hamsters, rabbits, and wild rabbits.

Screening Method to Provide Antagonistic Compounds Capable to Inhibit AM Functions.

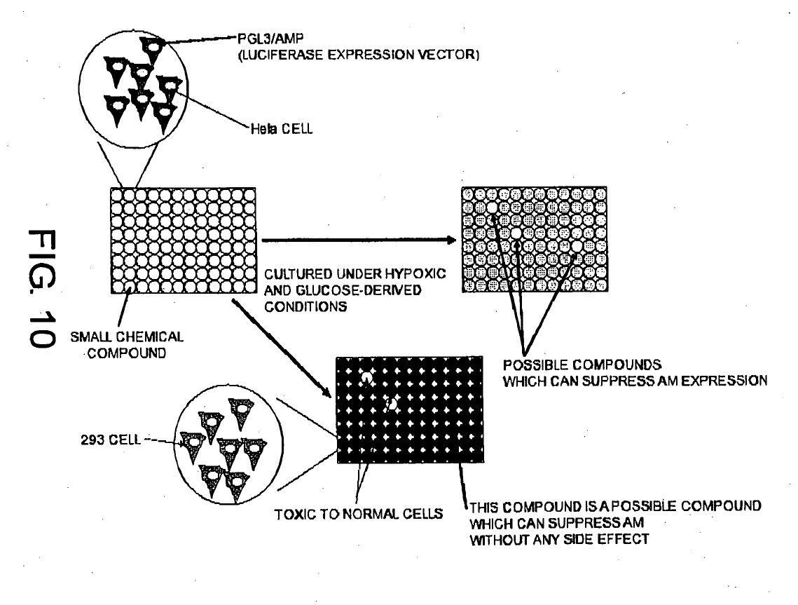

Promoter region of adrenomedullin (1.5 kb) was cloned using the registered sequence of adrenomedullin and by referencing to whole DNA sequences. This region was ligated into PGL3 and then used for luciferase assay (FIG. 10). Hela cells were transfected with PGL3/AMP. The cells were placed in wells of 96 well plates at 5×10⁴ and then cultured with small chemical compounds selected from a chemical library for 24 hr. Luciferase activity was determined by a Luminometer. For positive control, the cells were cultured under hypoxic and glucose-deprived conditions without additives. Given the elicitation of functional activity of AM solely dependent on the binding of AM on its specific receptor(s), the present invention involve a competitive receptor binding inhibition assay of labeled adrenomeddullin to target cells expressing its specific receptors. The procedures of the competitive receptor binding assay co can be carried out according to methods known in the art.

So far adrenomedullin receptor(s) is deemed to signal through Gs and adenylate cyclase, with Gq-activated increases in intracellular calcium levels being a less common second pathway. Adrenomedullin receptors follow this model, with increased intracellular cAMP being the usual result of an adrenomedullin challenge. In transfected cells CRLR/RAMP2 (specific adrenomedullin receptors) and CRLR/RAMP1 (CGRP1 receptors) combinations also increase intracellular cAMP levels. The difficulty in native cells and tissues is to determine which CRLR/RAMP combination is responsible for adrenomedullin signalling.

In order to skip this problem, cell lines expressing endogenous adrenomedullin binding. Three cell lines to date are available. L6 cells are a rat myoblast cell line known to express CGRP1 receptors, Swiss 3T3 cells are a mouse fibroblast cell line, and Rat-2 cells are rat fibroblasts. L6 cells were shown to express high levels of both adrenomedullin and CGRP binding. L6 cells express CRLR and RAMPs 1 and 2. In Swiss 3T3 cells the effects of adrenomedullin were mediated via specific adrenomedullin receptors. In Rat-2 cells we also were able to show dose-dependent stimulation of intracellular cAMP by adrenomedullin in the absence of CGRP binding Mitogen-activated protein kinases (MAPKS) are involved in control of cell growth, and can be activated in these cells by platelet-derived growth factor (PDGF). Adrenomedullin potently and dose-dependently inhibited PDGF-stimulated and basal MAPK activity. These cells are useful in establishing the screening system to inhibit functionally the intracellular signal transduction after a receptor engagement by adrenomedullin.

Non-peptidic compounds that regulate adrenomedullin angiogenic functions are so far not known. The method disclosed in the present invention provides an efficient way to screen a large library of small molecules and will be useful to the identification of positive and negative modulators of AM functions.

Methods of Treating Cardiovascular and Renal Diseases and Pharmaceutical Components:

In an alternate embodiment, pharmaceutical compositions of the present invention may contain genes comprising DNA encoding adrenomedullin, capable to generate thick angiogenesis or vasculogenesis of more than 8 μm (in case of murine models). Pharmaceutical compositions of the present invention can be administered in a dosage form using either non-viral vector or viral vectors.

When using a non-viral vector, DNA encoding a peptide of the present invention can be introduced into cells or tissues using a recombinant expression vector prepared by incorporating the DNA into a commonly used gene expression vector employing techniques as described before. Examples of methods for introducing genes into cells include the calcium phosphate co-precipitation method and the DNA direct injection method using glass micropipette.

Additional examples of methods for introducing genes into tissues include gene transfer methods using internal type liposome, electrostatic type liposomes, HVJ-liposomes, and improved HVJ-liposomes (HVJ-AVE liposome); gene transfer methods mediated by receptors; methods for transferring DNA molecules together with carriers (metal particles) into cells using a particle gun; methods for directly transferring naked-DNA; and transfer methods mediated by positively charged polymers. Herein, suitable expression vectors include pCAGGS (Gene 108, 193-200(1991)), pBK-CMV, pcDNA3.1, and pZeoSV (Invitrogen, Stratagene).

Viral vectors suitable for administration are exemplified by recombinant adenoviruses and retroviruses. Specifically, genes can be introduced into cells using recombinant expression vectors comprising DNA encoding a peptide of the present invention incorporated into DNA viruses or RNA viruses, such as retrovirus, adenovirus, adeno-associated virus, herpes virus, vaccinia virus, poxvirus, poliovirus, Sindbis virus, Sendai virus, SV40, and human immunodeficiency virus (HIV), which have been made a virulent, to infect cells with these recombinant viruses.

Among the aforementioned viral vectors, the infection efficiency of adenovirus is known to be extremely high as compared to other viral vectors. From this point of view, the adenoviral vector system is preferably used.

Pharmaceutical compositions of the present invention can be introduced into patients by the in vivo method for directly introducing pharmaceutical compositions into the body of a patient, or by the ex vivo method for introducing pharmaceutical compositions into a certain type of cells extracted from a patient and then returning the cells back into the body of the patient.

When pharmaceutical compositions of the present invention are administered by the in vivo method, they can be administered through an appropriate route according to the target to be treated, i.e., cells, tissues, and target organs. For example, they may be administered intravenously, intra-arterially, subcutaneously, intracutaneously, intramuscularly, or directly into tissues where the target cancer lesions are localized.

Various pharmaceutical preparation forms (e.g., liquid) are suitable for the above-described administration forms and can be routinely adopted. The conditions follows the examples described for cancae patient treatment.

Further, in order to facilitate the localization of the gene into the vicinity of the affected region, a sustained release preparation (e.g., mini-pellet) can be prepared to be embedded near the affected region. It is also possible to continuously and slowly administer the gene to the affected area using an osmotic pump or the like.

Contents of DNA in the pharmaceutical preparations can be appropriately adjusted according to the disease being treated, the age and body weight of the patient, etc.

Pharmaceutical compositions containing genes comprising DNA encoding adrenomedullin of the present invention can be used to treat various cardiovascular and renal diseases, including, but not limited to, congestive heart failure, myocardial infarction, hypertension, chronic renal failure, stroke, diabetes mellitus, and septic shock.

Following introduction into cells, the pharmaceutical compositions of the present invention are believed to produce adrenomedullin that generate macroangiogenesis of blood vessels and facilitate the impaired blood stream.

The blood vessel regenerating activity of pharmaceutical composition of the present invention can be assessed, for example, by generating iscemic conditions in hind limb in animals, such as rabbits, by administering the pharmaceutical composition, and examining changes in the size of blood vessels and blood stream.

Hereinafter, the present invention will be explained in more detail with reference to the Examples, but it should not be construed as being limited thereto.

### EXAMPLES

### Example 1

As noted above, it has been demonstrated that the disruption of adrenomedullin (AM) induced growth inhibition in several cancer cell lines. Accordingly, the present inventors hypothesized that AM is expressed in tumor tissues exposed to hypoxic and glucose-deprived conditions. Therefore, the expression of AM mRNA was examined in a variety of cancer cell lines under normoxic, hypoxic and/or glucose-deprived conditions.

### Example 1A

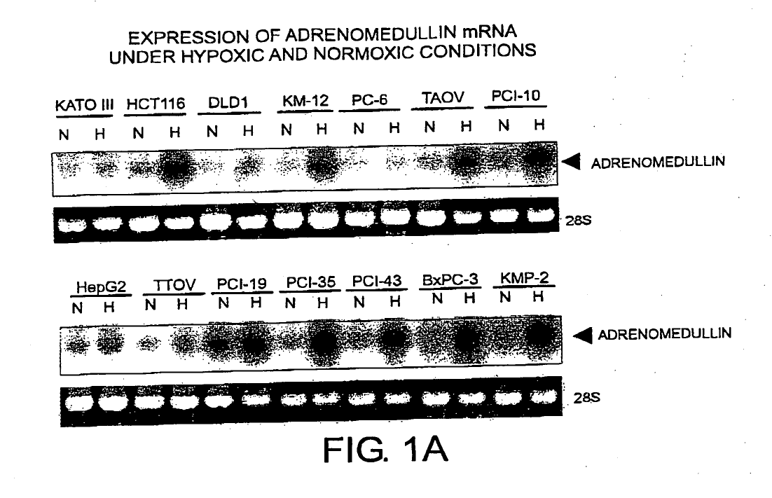

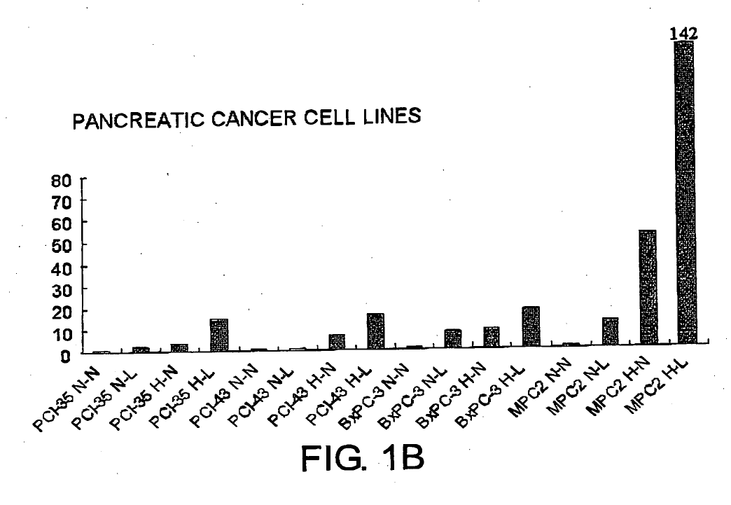

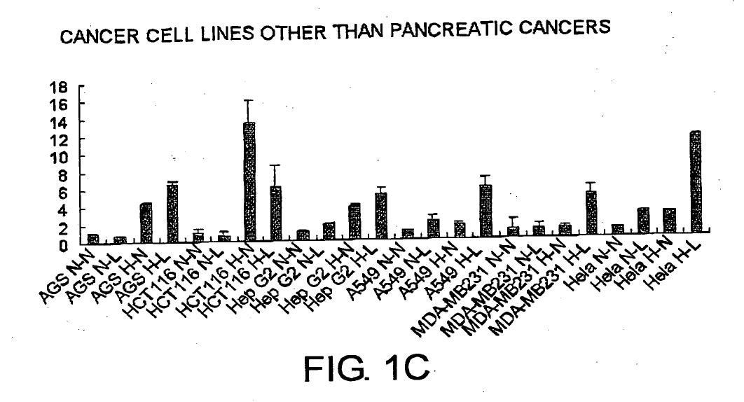

First, various cancer cell lines were cultured under hypoxic conditions to examine the expression of adrenomedullin (AM) mRNA by the Northern blot method. The culture of cancer cell lines under hypoxic conditions was carried out in 1% O₂ concentration for 12 h using a hypoxic culture chamber (Wakenyaku Industry). Culture in 20% O₂ concentration was also performed as a control. After the culture, RNA was extracted from various cancer cell lines using the TRIZOL reagent (LIFE TECHNOLOGIES). RNA (20 μg) was electrophoresed on formaldehyde-agarose gel, and then hybridized with an AM-specific probe. Results are shown in FIG. 1A.

Cancer cell lines used herein were as follows:

| | |
|---|---|
| KATO III | Stomach cancer cell line |
| HCT116 | Colorectal cancer cell line |
| DLD1 | Colorectal cancer cell line |
| KM-12 | Colorectal cancer cell line |
| PC-6 | Lung cancer cell line |
| TAOV | Ovarian cancer cell line |
| PCI-10 | Pancreatic cancer cell line |
| HepG2 | Liver cancer cell line |
| TTOV | Ovarian cancer cell line |
| PCI-19 | Pancreatic cancer cell line |
| PCI-35 | Pancreatic cancer cell line |
| PCI-43 | Pancreatic cancer cell line |
| BxPC-3 | Pancreatic cancer cell line |
| KMP-2 | Pancreatic cancer cell line |

FIG. 1A depicts the results of the Northern blot analysis of RNA extracted from various cancer cell lines, showing the 28S portion that reacted with the AM-specific probe. As shown in FIG. 1A, in cancer cells cultured under low oxygen (1% oxygen) concentrations (indicated with H in FIG. 1A), adrenomedullin mRNA increases compared to that in cancer cells cultured under normal oxygen (20% oxygen) concentration (indicated with N in FIG. 1A). This tendency was, in particular, conspicuous in pancreatic cancer cells.

### Example 1B

Next, various cancer cell lines were cultured under normoxic, hypoxic and/or glucose-deprived conditions. AM mRNA expression levels were examined using real time PCR. Results are shown in FIGS. 1B and 1C as relative copy numbers.

Pancreatic cancer cell lines were maintained in DMEM/F12 medium supplemented with 10% fetal calf serum (FCS, Filtron Pty Ltd., Australia). Other cancer cell lines were maintained in DMEM medium supplemented with 10% FCS. Cell lines were then cultured under one of the following conditions: normoxia with normal glucose (N—N); normoxia and glucose-deprived (N-L); hypoxia with normal glucose (H—N); or hypoxia and glucose-deprived (H-L). As above, normoxia comprised culture in 20% O₂ concentration and hypoxia comprised culture in 1% O₂ concentration for 12 h using a hypoxic culture chamber (Wakenyaku Industry). Normal glucose conditions involved addition of 100 mg/dl of glucose or higher whereas glucose-deprived conditions involved the addition of only 13 mg/dl glucose.

cDNA samples were extracted from each cell culture. Each cDNA (10 ng) was amplified in triplicates with the use of the SYBR-Green PCR assay kit and then detected on an ABI PRISM® 7900HT Sequence Detection System. The β-actin RNA was used to standardize the total amount of cDNA. The primers used were as follows:

| | |
|---|---|
| AM-GCTCCTCCTGAGCGCAAGT; | (SEQ ID NO:3) |
| and | |
| β-actin-TCGTCATACTCCTGCTTGCTGAT. | (SEQ ID NO:4) |

Relative mRNA levels were determined by comparing the PCR cycle thresholds between cDNA of the gene of interest and that of β-actin.

Results for pancreatic cancer cells lines PCI-35, PCI-43, BxPC-3 and MPC2 are shown in FIG. 1B. Results for other cancer cell lines, including AGS (gastric cancer cell line), HCT116 (colorectal cancer cell line), HepG2 (liver cancer cell line), MDA-MB231 (invasive breast cancer cell line), and HeLa (cervical cancer cell line), are shown in FIG. 1C. As expected, AM mRNA levels were higher under hypoxia (H) and under than under normoxia (N) in all cancer cell lines tested. Furthermore, AM mRNA levels were generally higher under hypoxic plus glucose-deprived conditions (H-L) than under either hypoxic (H—N) or glucose-deprived conditions alone (N-L). These results suggest that AM is highly expressed in tumor tissues, which manage to subsist under severe hypoxic and glucose-deprived conditions. Note, VEGF mRNA was expressed at higher levels under hypoxic condition, but not under hypoxic plus glucose-deprived conditions (results not shown).

### Example 2

On the dorsal side of a CB17lcr-scid Jl mouse (CLEA JAPAN Inc.), 10⁷ PCI-43 cells were subcutaneously transplanted. It was confirmed that PCI-43 cells proliferated to form a tumor, and that the tumor diameter exceeded 5 mm after 7 days. After that confirmation, 50 μg 35 each of adrenomedullin and a peptide of the present invention (dissolved in 0.1 ml of physiological saline) was injected into the tumor once a day starting on the 7^{th} day until the 16^{th} day. A peptide consisting of SEQ ID NO: 2 was used as the peptide of the present invention (hereinafter also referred to as an “adrenomedullin antagonist (AMA)”). This peptide was purchased from Wako Pure Chemical Industries, Ltd.

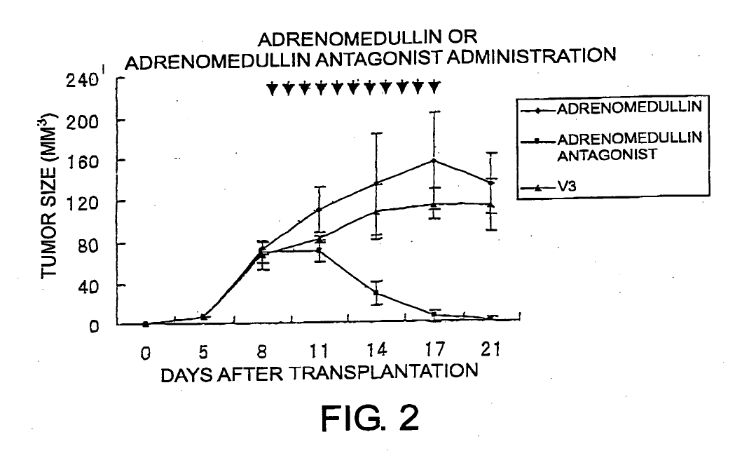

The tumor diameter was megascopically measured every three days. Results are shown in FIG. 2, a graphical representation of tumor sizes when adrenomedullin, adrenomedullin antagonist, and physiological saline (V3), respectively, were administered. The horizontal axis represents the number of days after transplanting PCI-43 cells, and the vertical axis the size of the tumor in volume (mm³). Arrows at the upper part of the graph indicate timings of administration of adrenomedullin, adrenomedullin antagonist, and physiological saline. Five CB 17lcr-scid Jl mice were used in each group.

As clearly shown in FIG. 2, in the group in which the adrenomedullin antagonist was administered from the 7^{th} day to 16^{th} day, the tumor size became smaller and on the 21^{st} day became almost megascopically invisible. Conversely, in both the adrenomedullin- and physiological saline-administered groups, the tumor size hardly changed.

### Example 3

On the dorsal side of CB17lcr-scid Jl mice (CLEA JAPAN Inc.), 10⁷ PCI-43 cells were subcutaneously transplanted. It was confirmed that PCI-43 cells proliferated to form a tumor, and that the tumor diameter exceeded 5 mm after 7 days. After that confirmation, 50 μg each 2 0 of adrenomedullin and adrenomedullin antagonist (dissolved in 0.1 ml of physiological saline) was injected into the tumor once every three days from the 7^{th} day, that is, on the 7^{th}, 10^{th}, 13^{th}, and 16^{th} days. Adrenomedullin and adrenomedullin antagonist used herein were the same as those used in Example 1.

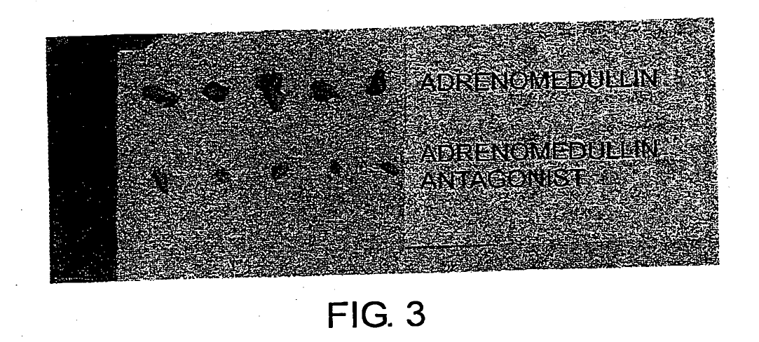

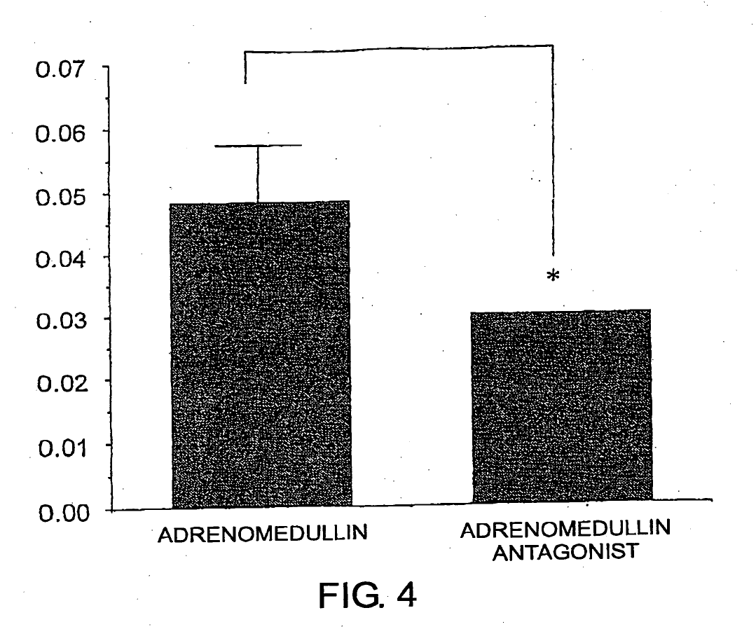

Mice were sacrificed on the 21^{st} day, and tumors were extirpated and weighed. A photograph of the extirpated tumors is shown in FIG. 3, and the results of tumor weighing are shown in FIG. 4.

As clearly seen from FIG. 3, the size of the tumor became smaller in the adrenomedullin antagonist-administered group compared to the adrenomedullin-administered group. Furthermore, as clearly shown in FIG. 4, the tumor weight was about 0.05 g in the adrenomedullin-administered group while that of the adrenomedullin antagonist-administered group was about 0.03 g, decreasing to about half of that of the adrenomedullin-administered group.

### Example 4

The effect of a peptide of the present invention on angiogenesis was examined. Experiments were carried out with the tumor tissues of mice used in Example 3. Mouse tumor tissues were extirpated, and CD31 antigen, which is the cell surface marker specific for vascular endothelial cells, was stained with anti-CD31 antibody. Staining was performed as described below. The experiments were carried out using five mice.

Tumor tissues were frozen in liquid nitrogen to prepare frozen sections. After the frozen sections were pre-treated in albumin solution for 30 min, endogenous peroxidase activity was suppressed with hydrogen peroxide, and then, the sections were treated with anti-CD31 antibody for 1 h at room temperature. After washing, the sections were treated with a secondary antibody for 1 h, and coloured using ECL (Amersham).

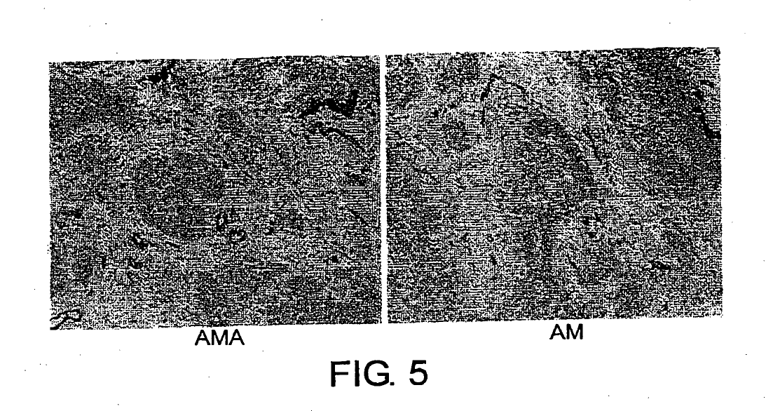

Results of staining are shown in FIG. 5. The staining results for the tumor tissue from an adrenomedullin-administered mouse are shown on the left side of FIG. 5, while those from an adrenomedullin antagonist-administered mouse are shown on the right side. In FIG. 5, the area stained brown represents the newly generated blood vessels. As clearly seen from FIG. 5, in the adrenomedullin-administered group, thick blood vessels were newly generated, while in the adrenomedullin antagonist-administered group, newly generated blood vessels were thin, demonstrating inhibition of angiogenesis.

Next, 100 newly generated blood vessels of the vascular endothelial cells used in Example 4 were arbitrarily selected to measure their respective diameters. In Table 1, results of the number of the newly generated blood vessels classified by their diameters into three groups (less than 2 μm, 2 μm to less than 8 μm, and 8 μm or more) are shown together with the average of blood vessel diameters and standard deviations. In Table 1, AM stands for adrenomedullin, and AMA adrenomedullin antagonist.

TABLE 1

| | Number of blood vessels | | | |
|---|---|---|---|---|
| | less than 2 | 2 μm to less | 8 μm or | |
| | μm | than 8 μm | more | Average ± standard deviation |
| AM | 15 | 54 | 31 | 6.454 ± 5.313 |
| AMA | 55 | 44 | 1 | 2.276 ± 1.120 |

As clearly shown in Table 1, in adrenomedullin antagonist-administered mice, the diameters of blood vessels became smaller, with almost no blood vessel having a particularly large diameter, further demonstrating inhibition of angiogenesis. In addition, in the adrenomedullin antagonist-administered mice, blood vessel diameters were one half of those of the adrenomedullin-administered mice.

### Example 5

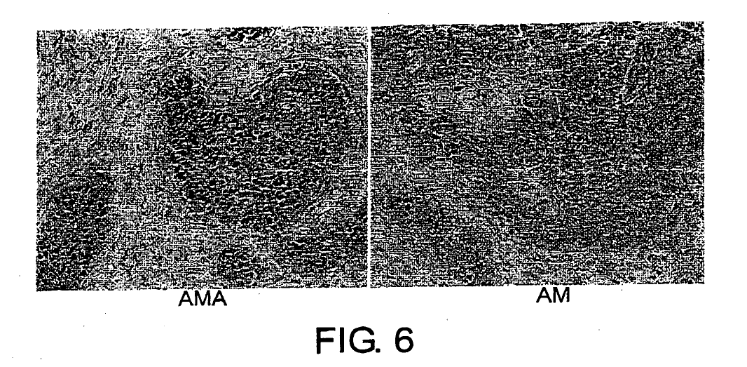

The effect of a peptide of the present invention on cell proliferation was examined. The proliferating cell nuclear antigen (PCNA) of tumor cells from the mice used in Example 2 was stained with anti-PCNA antibody. The staining method was the same as in Example 4. Five mice were used in the experiment. Results are shown in FIG. 6. The staining results for tumor cells of an adrenomedullin-administered mouse are shown on the left side in FIG. 6, while those of an adrenomedullin antagonist-administered mouse are shown on the right side.

As clearly seen from FIG. 6, many cells reacting with the anti-PCNA antibody were present in the adrenomedullin-administered group, while few cells reacted with the anti-PCNA antibody in the adrenomedullin antagonist-administered group. PCNA-labelling indexes were 25.8±3.9 and 6.3±5.2 in the adrenomedullin- and adrenomedullin antagonist-administered groups, respectively. Thus, proliferating cells decreased by about one quarter in the adrenomedullin antagonist-administered mice as compared to the adrenomedullin-administered mice.

As described above in detail, peptides of the present invention inhibit angiogenesis of cancer cells and suppress proliferation of the cells due to their inhibitory effect. Accordingly, peptides of the present invention find utility in treating various cancers, including, but not limited to, stomach cancer, colorectal cancer, lung cancer, ovarian cancer, liver cancer, and pancreatic cancer.

### Example 6

As demonstrated above, intra-tumoral injection of an AMA peptide suppressed the growth of pancreatic cancer cells in vivo. Herein, the effect of intra-tumoral and intramuscular injection of various AMA expression vectors on the in vivo growth of pancreatic and breast cancer cell lines is examined.

Mouse models were prepared according to the protocol described in Example 2 above. Specifically, 5×10⁶ cancer cells were inoculated subcutaneously into the right flanks of SCID mice (CB17lcr-scid Jl mouse (CLEA JAPAN Inc)) (n=5, in each group). Tumor formation was observed every three days up to 3 weeks after inoculation.

AMA expression vectors were prepared as follows:

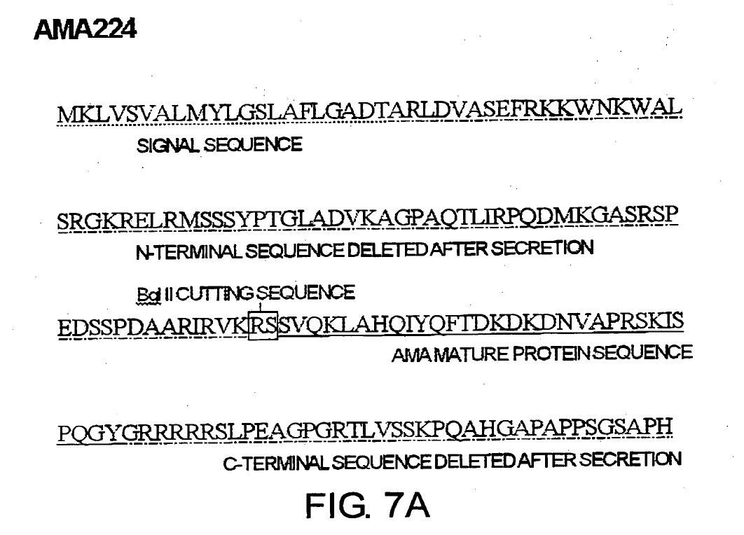

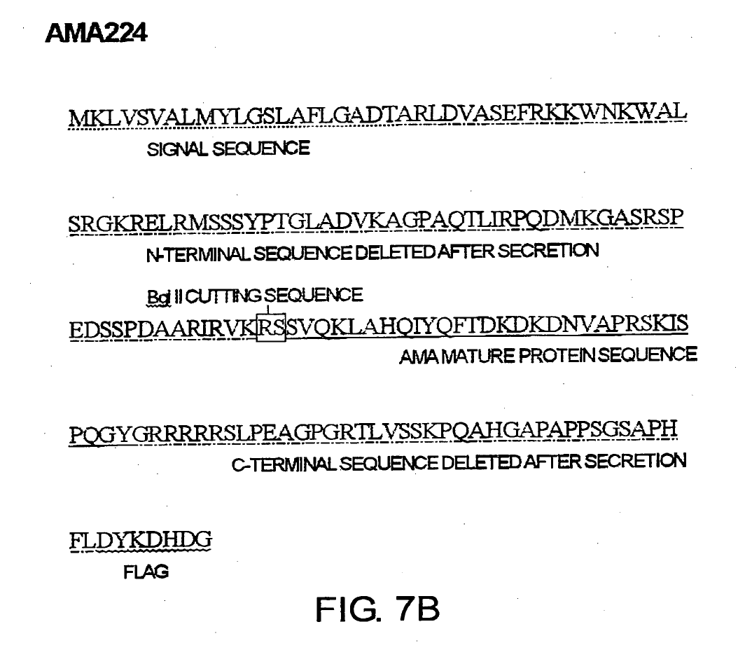

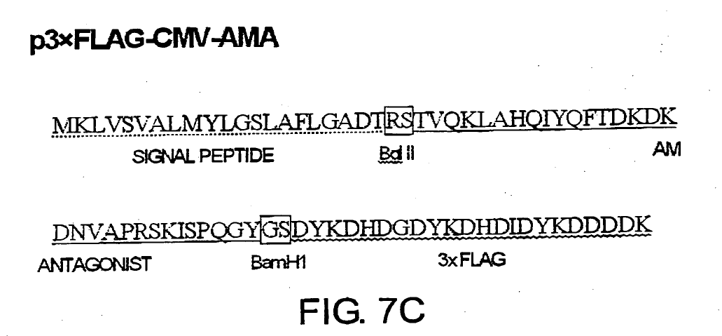

Full length of human adrenomedullin precursor cDNA was isolated by RT-PCR using total RNA from hypoxia-treated K562 cells. Three separate adrenomedullin antagonist (AMA) expression vectors were generated by PCR combined with site-directed mutagenesis. A first cDNA sequence, AMA214 (SEQ ID NO: 5), is shown in FIG. 7A and comprises the following domains: a signal peptide, a Bgl II recognition sequence, an AMA domain (mature protein sequence), and a C-terminal fragment plus three Flag tag repeats. AMA214 lacks amino acids 22-115 of the 185 amino acids adrenomedullin preprohormone (SEQ ID NO: 8; GenBank Accession No. NP_{—}001115) and contains a threonine:serine mutation at residue 116. A second cDNA sequence, AMA224 (SEQ ID NO: 6), is shown in FIG. 7B and comprises a signal peptide, a 20 amino acid residue N-terminal fragment, a Bgl II recognition sequence, an AMA domain (mature protein sequence), and a C-terminal fragment plus three Flag tag repeats. AMA224 lacks amino acids 95-115 of the 185 amino acids adrenomedullin preprohormone and contains a threonine:serine mutation at residue 116. Each cDNA, AMA214 and AMA224, was separately cloned into a p3xFLAG-CMV-14 plasmid (SIGMA), which carried three repeats of FLAG-tag at the C-terminus of the AMA domain, respectively. A third cDNA, p3xFLAG-CMV-AMA, is shown in FIG. 7C and comprises the following domains: a signal peptide, a Bgl II recognition sequence, an AMA domain (mature protein sequence), a Bam HI recognition sequence, and three Flag tags (SEQ ID NO: 7). p3xFLAG-CMV-AMA, was constructed by inserting the AMA cDNA into a p3xFLAG-CMV-14 plasmid as described above. Empty vectors, either p3xFLAG or pcDNA3.1-FLAG, were used as a control.

### Example 6A

The effect of intra-tumoral injection of AMA expression vectors on the in vivo growth of pancreatic cancer cells is examined herein.

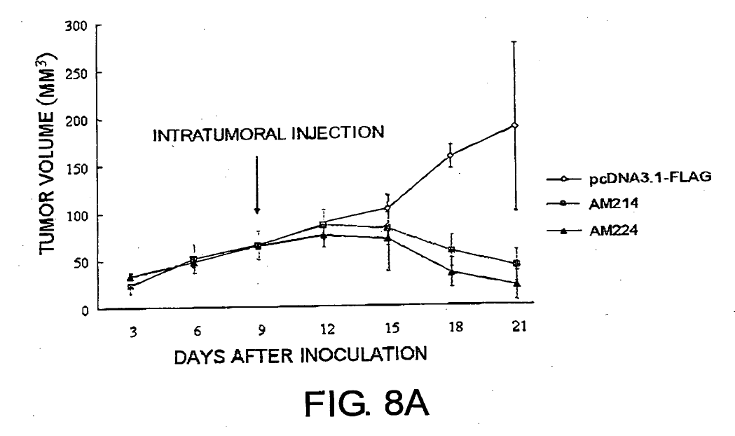

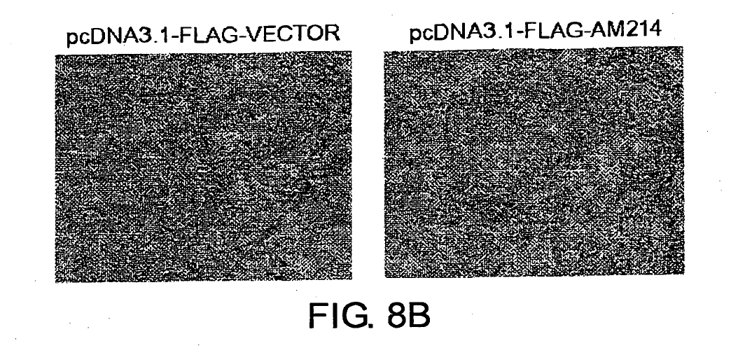

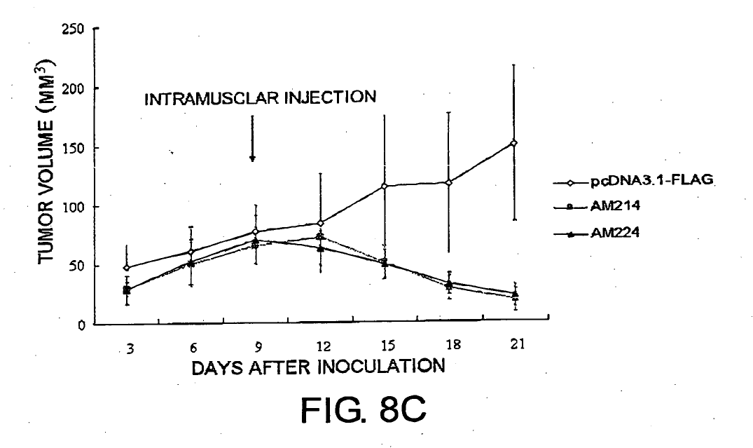

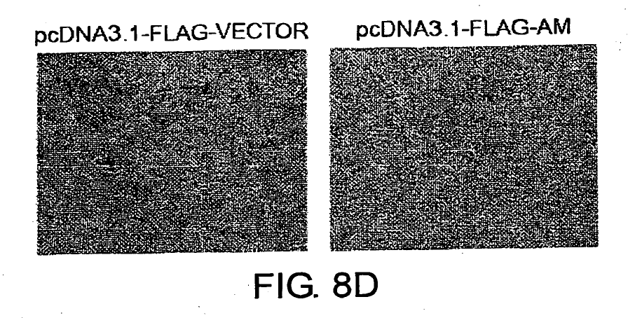

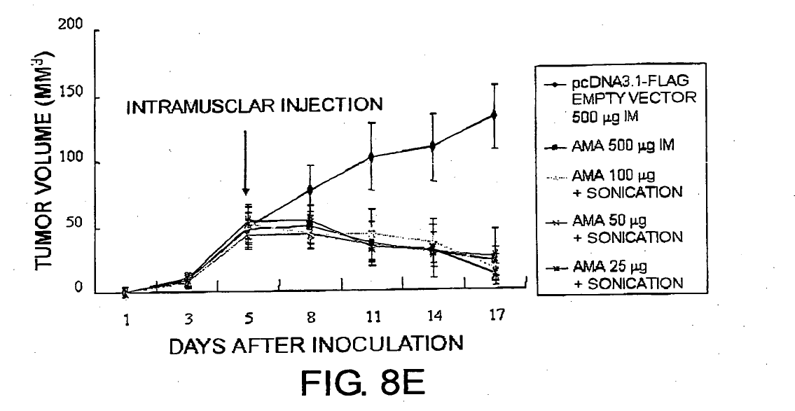

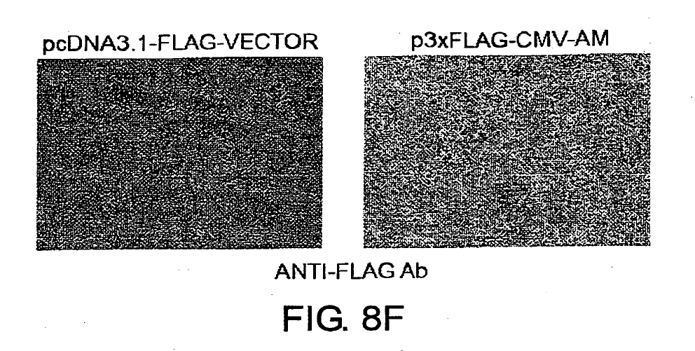

Five hundred (500) μg of either AMA214, AMA224 or an empty expression vector(pcDNA3.1-FLAG) serving as a control were injected intra-tumorally 9 days after subcutaneous inoculation of pancreatic cancer cells (PCI-43) into the back of SCID mice. Growth curves are depicted in FIG. 8A. Whereas the control vector had no effect on growth of cancer cells, intra-tumoral injection of the AMA214 and AMA224 expression vectors induced regression of implanted pancreatic cancer cells.

Twenty-one days after inoculation, mouse tumor tissues were extirpated. Expression of CD31 was analyzed by immunohistochemical staining using the streptavidin-biotin technique (Histofine SAB-PO kit, Nichirei, Tokyo). Snap-frozen tissue specimens were used for the analysis. The tissue sections were pre-incubated for 30 minutes with PBS containing 1% bovine serum albumin and inactivated with 3% H₂O₂ in methanol for 15 minutes. The sections were then incubated overnight at 4° C. with anti-mouse CD31 antibody (BD Pharmingen, San Diego, Calif.) at a concentration of 5 μg/ml in PBS. After washing with PBS, the sections were incubated for 1 h with the biotin-conjugated anti-rat second antibody (DAKO, Tokyo), followed by the avidin-biotin-peroxidase reaction. DAB was used as a chromogen to visualize the reaction products. Finally, all the sections were counter-stained with hematoxylin. Immunocytochemical staining for vector was performed, using anti-Flag antibody (Peninsula Laboratories INC., San Carlos, Calif.) and a Histofine simple staining PO kit (Nichirei, Tokyo), according to the manufacturer's instructions.

FIG. 8B shows that CD31-positive cells were observed only in the tumor tissues treated with control vector, but not in the tumor tissues treated with the AMA214 expression vector. These results indicate that angiogenesis is inhibited in AMA treated cells.

### Example 6B

The effect of intramuscular injection of AMA expression vectors on the in vivo growth of pancreatic cancer cells is examined herein.

Five hundred (500) μg of either pcDNA3.1-FLAG-AMA, AMA214, AMA224 or an empty expression vector (pcDNA3.1-FLAG) was injected into the femoral muscle of SCID mice 9 days after subcutaneous inoculation of pancreatic cancer cells (PCI-43). Growth curves for AMA214, AMA224 and the control vector (pcDNA3.1-FLAG) are depicted in FIG. 8C. Whereas the control vector had no effect on growth of cancer cells, injection of the AMA214 and AMA224 expression vectors into the distant muscle induced the regression of implanted pancreatic cancer cells.

Twenty-one days after inoculation, mouse tumor tissues were extirpated and incubated with anti-mouse CD31 antibody according to the protocol discussed above in Example 6A. FIG. 8D shows that CD31-positive cells were observed only in the tumor tissues treated with the control vector, pcDNA3.1-FLAG, but not in the tumor tissues treated with the pcDNA3.1 -FLAG-AMA expression vector. These results indicate that angiogenesis is inhibited in AMA treated cells.

### Example 6C

The effect of intramuscular injection of various concentrations of an AMA expression vector on the in vivo growth of a breast cancer cells is examined herein.

Five hundred (500) μg of an empty vector (p3xFLAG) serving as a control were injected into the femoral muscle of SCID mice 9 days after subcutaneous inoculation of breast cancer cells (MDA-MB-231). Experimental groups were dosed with either 500 μg, 100 μg, 50 μg, or 25 μg of the AMA expression vector, p3xFLAG-CMV-AMA. Sonication was added to the latter three experimental groups to enhance expression efficiency. Growth curves are depicted in FIG. 8E. Whereas the control vector had no effect on growth of cancer cells, injection of the AMA expression vector, p3xFLAG-CMV-AMA, into the distant muscle suppressed the growth of MDA-MB-231 cells.

Twenty-one days after inoculation, mouse tumor tissues were extirpated and incubated with anti-mouse CD31 antibody according to the protocol discussed above in Example 6A. Tissues were simultaneously incubated with anti-adrenomedullin antibody (BD Pharmingen, San Diego, Calif.), also at a concentration of 5 μg/ml in PBS.

The upper panels of FIG. 8F demonstrate that CD31-positive cells were observed only in the tumor tissues treated with the control vector, but not in the tumor tissues treated with the AMA expression vector, p3xFLAG-CMV-AMA. In addition, adrenomedullin (AM) was found to be expressed in the tumor cells treated with or without AMA (results not shown). The lower panels of FIG. 8F demonstrate that flag-tagged AMA was observed only in the tumor tissues treated with the p3xFLAG-CMV-AMA expression vector.

These results suggest that AMA suppresses the growth of cancer cells through the suppression of AM-induced angiogenesis. As angiogenesis is regulated by multiple factors, many reports have concluded that suppression of one angiogenic factor is not enough for the complete disappearance of blood vessels in tumor tissues. However, the in vivo results herein demonstrate that suppression of only one angiogenic factor, adrenomedullin (AM), completely suppressed angiogenesis.

### Example 7

In an effort to explain the mechanisms by which AMA alone could suppress angiogenesis, it was proposed that perhaps adrenomedullin is not an endothelial growth factor but instead is an essential factor for the survival of endothelial cells under hypoxic and nutrient-deprived conditions (i.e., tumor conditions). Accordingly, the effects of AM and AMA on the growth and apoptosis of endothelial cells under various culture conditions and in the presence of various mediating factors were examined.

### Example 7A

The effect of AM on the growth of endothelial cells under normal conditions (normoxia, normal glucose) is examined herein. As in Example I above, normoxic conditions comprised culture in 20% O₂ concentration and normal glucose conditions involved addition of 100 mg/dl of glucose or higher.

Five×10³ cells (2×10⁴ in some experiments) were plated in wells of 96-well plates. Human adrenomedullin (AM) (SEQ ID NO: 1) was added at indicated concentrations, ranging from 0 to 10⁻⁶ M. The cells were incubated for 48 h and the viable cell numbers were determined by MTS assay according to the manufacturer's instructions. Cell viability can be reflected by the integrity of the mitochondria. When the MTS reagent (a tetrazolium salt) is applied to living cells, it is converted to a color compound (formazan) with the emission of light at 490 nm. Accordingly, the MTS assay may be used to assay cell viability and to count viable cells.

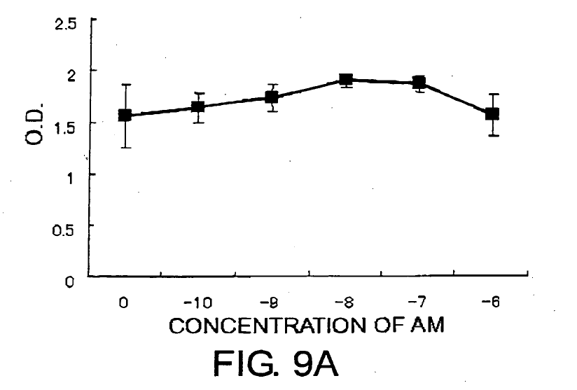

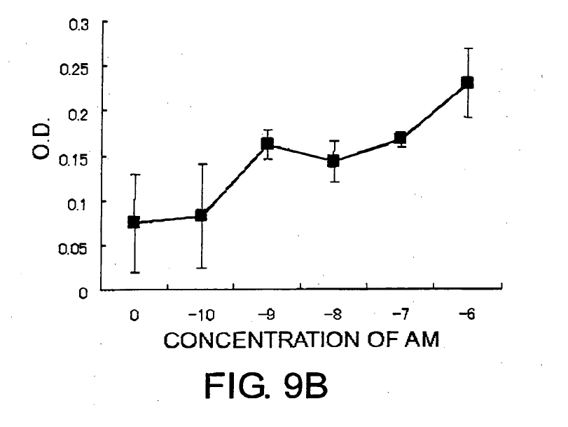

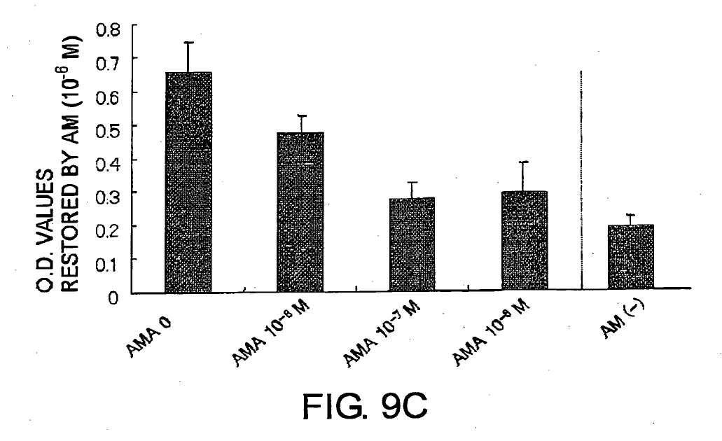

Results are shown in FIG. 9A. The x-axis represents concentrations of AM and the y-axis represents optical density (O.D.). As FIG. 9A demonstrates, AM had no significant effect on the growth of endothelial cells under normoxic and nutrient-supplied conditions. Accordingly, it appears that adrenomedullin is an endothelial cell growth factor.

### Example 7B

The effect of AM on the growth and apoptosis of endothelial cells under extreme conditions (hypoxic and nutrient-deprived) and in the presence of growth factors is examined herein. As in Example 1 above, hypoxic conditions comprised culture in 1% O₂ concentration for 12 h using a hypoxic culture chamber (Wakenyaku Industry) and nutrient-deprived conditions involved addition of only 13 mg/dl glucose.

Five×10³ cells (2×10⁴ in some experiments) were plated in wells of 96-well plates. Human adrenomedullin (AM) (SEQ ID NO: 1) was added at indicated concentrations, ranging from 0 to 10⁻⁶ M. VEGF and FGF were added at 10 ng/ml. The cells were incubated for 48 h and the viable cell numbers were determined by the MTS assay according to the manufacturer's instructions as described above in Example 7A.

Results are shown in FIG. 9B. Again, the x-axis represents concentrations of AM and the y-axis represents optical density (O.D.). As FIG. 9B demonstrates, under hypoxic and glucose-deprived conditions, endothelial cells underwent apoptosis, even in the presence of VEGF and FGF. However, AM protected the endothelial cells from apoptosis that was induced by the hypoxic and glucose-deprived conditions. These results suggest that AM is essential for the survival of the endothelial cells under hypoxic and nutrient-deprived conditions, even in the presence of VEGF or FGF.

### Example 7C

The effect of both AM and AMA together on the growth and apoptosis of endothelial cells under extreme conditions (hypoxic and nutrient-deprived) and in the presence of growth factors is examined herein. As in Example 1 above, hypoxic conditions comprised culture in 1% O₂ concentration for 12 h using a hypoxic culture chamber (Wakenyaku Industry) and nutrient-deprived conditions involved addition of only 13 mg/dl glucose.

Five×10³ cells (2×10⁴ in some experiments) were plated in wells of 96-well plates. Human adrenomedullin (AM) (SEQ ID NO: 1) was added at a concentration of 10⁻⁶ M and a human adrenomedullin antagonist peptide (AMA) (SEQ ID NO: 2) was added at indicated concentrations, ranging from 0 to 10⁻⁶ M. VEGF and FGF were added at 10 ng/ml. The cells were incubated for 48 h and the viable cell numbers were determined by the MTS assay according to the manufacturer's instructions as described above in Example 7A.

Results are shown in FIG. 9C. The x-axis represents concentrations of AMA and the y-axis represents optical density (O.D.). As FIG. 9C demonstrates, AMA induced apoptosis of the endothelial cells in a dose dependent manner, even in the presence of AM. These results suggest that AMA can inhibit the protective effect of AM and induce apoptosis even in the presence of AM under hypoxic and nutrient-deprived conditions.

Collectively, all these results suggest that AM is essential for angiogenesis in tumor tissues and that the interruption of angiogenesis by AMA is effective for the treatment of solid tumors.

### Example 8

### siRNA Production

Three sequences of siRNAs for adrenomedulin were selected as shown below. All these siRNAs suppressed the expression of adrenomedullin mRNA and adrenomedullin protein in PCI-43 and MDA-MB231 cell lines.

The cells were treated with 5 nM siRNA for AM for 24 hr. As a control siGFP was used. SiRNAs for AM but siGFP significantly suppressed expression of AM mRNA(around 92%) and protein.

siRNA for AM

template used for the production of siRNA for AM

| | |
|---|---|
| Antisense siRNA Oligonucleotide Template: | |
| 5′-AAGCTGGCACACCAGATCTACCCTGTCTC-3′ | (SEQ ID NO:9) |
| Sense siRNA Oligonucleotide Template: | |
| 5′-AAGTAGATCTGGTGTGCCAGCCCTGTCTC-3′ | (SEQ ID NO:10) |
| Antisense siRNA Oligonucleotide Template: | |
| 5′-AAGCTGGTTTCCGTCGCCCTGCCTGTCTC-3′ | (SEQ ID NO:11) |
| Sense siRNA Oligonucleotide Template: | |
| 5′-AACAGGGCGACGGAAACCAGCCCTGTCTC-3′ | (SEQ ID NO:12) |
| Antisense siRNA Oligonucleotide Template: | |
| 5′-AAGCGCTACCGCCAGAGCATGCCTGTCTC-3′ | (SEQ ID NO:13) |
| Sense siRNA Oligonucleotide Template: | |
| 5′-AACATGCTCTGGCGGTAGCGCCCTGTCTC-3′ | (SEQ ID NO:14) |

### Example 9

### Screening of AM Inhibitor; Luciferase Assay

Promoter region of adrenomedullin (1. 5 kb) was cloned using the registered sequence of adrenomedullin and by referencing to whole DNA sequences. This region was ligated into of PGL3 and then used for luciferase assay (FIG. 10). Hela cells were transfected with 10 ng of PGL3/AMP with the use of a Lipofectamine method. After transfection the cells were placed in wells of 96 well plates at 5×10⁴ and then cultured with small chemical compounds selected from a chemical library for 24 hr. Luciferase activity was determined by a Luminometer. For positive control, the cells were cultured under hypoxic and glucose-deprived conditions without additives.

### Example 10

### Establishment of an Expression Vector of Adrenomedullin Using a Non-Viral Plasmid Vector

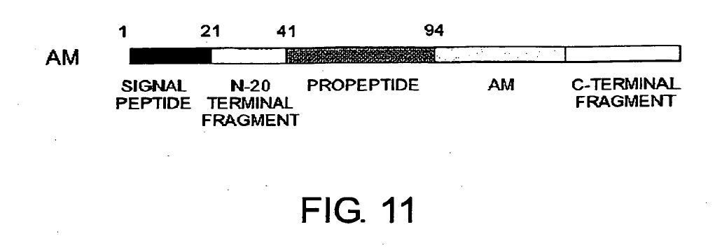

Full length of adrenomedullin cDNA (shown in FIG. 11) was ligated into ECOR1 site of pcDNA3.1 expression vector. AM expression vectors were prepared as follows: Full length of human adrenomedullin precursor cDNA was isolated by RT-PCR using total RNA from hypoxia-treated K562 cells.

The blood vessel regenerating activity of pharmaceutical composition of the present invention was assessed by generating iscemic conditions in hind limb in rabbits, by administering the expression vector every two weeks in the femoral muscle after femoral artery resection and by examining changes in the size of blood vessels and blood stream. When the expression vector, large blood vessels more than 8 μm dimers were formed in the femur, whereas no detectable large blood vessels was visible by immunohisotochemistry.

### INDUSTRIAL APPLICABILITY

Adrenomedullin antagonist (AMA) peptides are demonstrated herein to inhibit angiogenesis of cancer cells, suppress the proliferation of cancer cells due to such an inhibitory effect, and suppress the protective activity of adrenomedullin. Accordingly, peptides and pharmaceutical compositions of the present invention described herein find utility in the treatment of various cancers, including, but not limited to, stomach cancer, colorectal cancer, lung cancer, ovarian cancer, liver cancer, and pancreatic cancer.

All publications, patent applications, patents, and other references mentioned herein are incorporated by reference herein in their entirety.

### ADDITIONAL REFERENCES

Akakura N, Kobayashi M, Horiuchi I, Suzuki A, Wang J, Chen J, Niizeki H, Kawamura K, Hosokawa M, and Asaka M. (2001) Constitutive expression of hypoxia-inducible factor-1α (HIF-1α) renders pancreatic cancer cells resistant to apoptosis induced by hypoxia and nutrient deprivation. Cancer Res., 61, 6548-6554.
Browder T, Folkman J, and Pirie-Shepherd S. (2000) The homeostatic system as a regulator of angiogenesis. J Biol. Chem., 275, 1521-1524.
Carmeliet P, Dor Y, Herbert J M, Fukumura D, Brusselmans K, Dewerchin M, Neeman M, Bono F, Abramovitch R, Maxell P, Koch C J, Ratcliffe P, Moons F, Jain R K, Collen D, and Keshet E. (1998) Role of HIF-1α in hypoxia-mediated apoptosis, cell proliferation and tumor angiogenesis. Nature, 394, 485-490
Carmeliet P. (2000) Mechanisms of angiogenesis and arteriogenesis. Nat. Med., 6, 389-395.
Choi S, Kobayashi M, Wang J, Habellhah H, Okada F, Hamada J, Moriuchi T, Totsuka Y, and Hosokawa M. (2000) Activated leukocyte cell adhesion molecule (ALCAM) and annexin II are involved in the metastatic progression of tumor cells after chemotherapy with adriamycin. Clin. Exp. Metastasis, 18,45-50.
Cormier-Regard S, Nguyen S V, Claycomb W C. (1998) Adrenomedullin gene expression is developmentally regulated and induced by hypoxia in rat ventricular cardiac myocytes. J. Biol. Chem., 273, 1787-1792.
Drake C J, Little C D. (1995) Exogenous vascular endothelial growth factor induces malformed and hyperfused vessels during embryonic neovascularization. Proc. Natl. Acad. Sci. USA., 92, 7657-7661.
Eguchi S, Hirata Y, Iwasaki H, Sato K, Watanabe T X, Inui T, Nakajima K, Sakakibara S, Marumo F. (1994) Structure-activity relationship of adrenomedullin, a novel vasodilatory peptide, in cultured rat vascular smooth muscle cells. Endocrinology, 135, 2454-2458.
Folknan J. (1995) Angiogenesis in cancer, vascular, rheumatoid and other disease. Nat. Med., 1, 27-31.
Garayoa M, Martinez A, Lee S, Pio R, An W C, Neckers L, Trepel J, Montuenga L M, Ryan H, Johnson R, Gassmann M, Cuttitta F. (2000) Hypoxia-inducible factor-1 (HIF-1) up-regulates adrenomedullin expression in human tumor cell lines during oxygen deprivation: a possible promotion mechanism of carcinogenesis. Mol. Endocrinol., 14, 848-862.
Hayakawa H, Hirata Y. Kakoki M, Suzuki Y. Nishimatsu H, Nagata D, Suzuki E, Kikuchi K, Nagano T, Kangawa K, Matsuo H, Sugimoto T, Omata M. (1999) Role of nitric oxide-cGMP pathway in adrenomedullin-induced vasodilation in the rat. Hypertension, 33, 689-693.
Kato H, Shichiri M, Marumo F, Hirata Y (1997) Adrenomedullin as an autocrine/paracrine apoptosis survival factor for rat endothelial cells. Endocrinology, 138, 2615-2620.
Kerbel R S. (2000) Tumor angiogenesis: past, present and the near future. Carcinogenesis, 21, 505-515.
Miller M J, Martinez A, Unsworth E J, Thiele C J, Moody T W, Elsasser T, Cuttitta F. (1996) Adrenomedullin expression in human tumor cell lines. Its potential role as an autocrine growth factor. J Biol. Chem., 271, 23345-51
Muff R, Born W, and Fischer J A. (2001) Adrenomedullin and related peptides: receptors and accessory proteins. Peptides, 22, 1765-1772.
Nakayama M, Takahashi K, Murakami O, Shirato K, Shibahara S. (1998) Induction of adrenomedullin by hypoxia and cobalt chloride in human colorectal carcinoma cells. Biochem. Biophys. Res. Commun., 243, 514-517.
Niizeki H, Kobayashi M, Horiuchi I, Akakura N, Chen J, Wang J, Hamada J, Seth P, Katoh H, Watanabe H, Raz A, and Hosokawa M. (2002) Hypoxia enhances the expression of autocrine motility factor and the motility of human pancreatic cancer. Br J Cancer, 86, 1914-1919.
Nuki C, Kawasaki H, Kitamura K, Takenaga M, Kangawa K, Eto T, Wada A. (1993) Vasodilator effect of adrenomedullin and calcitonin gene-related peptide receptors in rat mesenteric vascular beds. Biochem. Biophys. Res. Commun., 196, 245-251.
Oehler M K, Norbury C, Hague S, Rees M C, Bicknell R. (2001) Adrenomedullin inhibits hypoxic cell death by upregulation of Bcl-2 in endometrial cancer cells: a possible promotion mechanism for tumour growth. Oncogene, 20, 2937-2945.
Oehler M K, Hague S, Rees M C, and Bicknell R. (2002) Adrenomedullin promotes formation of xenografted endometrial tumors by stimulation of autocrine growth and angiogenesis. Oncogene, 21, 2815-2821.
Ouafik L, Sauze S, Boudouresque F, Chinot O, Delfmo C, Fina F, Vuaroqueaux V, Dussert C, Palmari J, Dufour H, Grisoli F, Casellas P, Brunner N, and Martin P M. (2002) Neutralization of adrenomedullin inhibits the growth of human glioblastoma cell lines in vitro and suppresses tumor xenograft growth in vivo. Am J Pathol. 160, 1279-1292.
Rocchi P, Boudouresque F, Zamora A J, Muracciole X, Lechevallier E, Martin P M, Ouafik L. (2001) Expression of adrenomedullin and peptide amidation activity in human prostate cancer and in human prostate cancer cell lines. Cancer Res., 61, 1196-1206.
Suri C, McClain J, Thurston C G McDonald D M, Zhou H, Oldmixon E H, Sato T N, and Yancopoulos G D. (1998) Increased vascularization in mice overexpressing angiopoietin-1. Science, 282, 468-471.
Watanabe T X, Itahara Y, Inui T, Yoshizawa-Kumagaye K, Nakajima K, Sakakibara S. (1996) Vasopressor activities of N-terminal fragments of adrenomedullin in anesthetized rat. Biochem. Biophys. Res. Commun., 219, 59-63.
Yancopoulos G D, Davis S, Gale N W, Rudge J S, Wiegand S J, and Holash J. (2000) Vascular-specific growth factors and blood vessel formation. Nature, 407, 242-248.
Zhao L, Brown L A, Owji A A, Nunez D J, Smith D M, Ghatei M A, Bloom S R, Wilkins M R. (1996) Adrenomedullin activity in chronically hypoxic rat lungs. Am. J Physiol., 271, 622-629.

Unless otherwise defmed, all technical and scientific terms used herein have the same meaning as commonly understood by one of ordinary skill in the art to which this invention belongs. In case of conflict, the present specification, including definitions, will control. In addition, the materials, methods, and examples are illustrative only and not intended to be limiting. Methods and materials similar or equivalent to those described herein can be used in the practice or testing of the present invention.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1-33. (canceled)

34. An isolated nucleic acid encoding a truncated peptide, wherein said truncated peptide comprises the amino acid sequence set forth in SEQ ID NO:1 in which at least one but no more than twenty-five amino acids are deleted from the N-terminal side, wherein said truncated peptide inhibits the finction of adrenomedullin to generate macroangiogenesis.

35. The isolated nucleic acid of claim 34, wherein said truncated peptide comprises the amino acid sequence set forth in SEQ ID NO:1 in which at least one but no more than twenty amino acids are deleted from the N-terminal side.

36. The isolated nucleic acid of claim 34, wherein said truncated peptide comprises the amino acid sequence set forth in SEQ ID NO:1 in which at least one but no more than fifteen amino acids are deleted from the N-terminal side.

37. The isolated nucleic acid of claim 34, wherein said truncated peptide comprises the amino acid sequence set forth in SEQ ID NO:1 in which at least one but no more than ten amino acids are deleted from the N-terminal side.

38. The isolated nucleic acid of claim 34, wherein said truncated peptide comprises the amino acid sequence set forth in SEQ ID NO:1 in which at least one but no more than five amino acids are deleted from the N-terminal side.

39. The isolated nucleic acid of claim 34, wherein said truncated peptide further comprises a mutation, wherein said mutation is selected from the group consisting of a deletion, a substitution, and an insertion.

40. A pharmaceutical composition for inhibiting tumor angiogenesis comprising a compound capable of inhibiting the finction of adrenomedullin to generate macroangiogenesis.

41. The pharmaceutical composition of claim 40, wherein said compound is a truncated peptide, wherein said truncated peptide comprises the amino acid sequence set forth in SEQ ID NO:1 in which at least one but no more than twenty-five amino acids are deleted from the N-terminal side.

42. The pharmaceutical composition of claim 41, wherein said truncated peptide comprises the amino acid sequence set forth in SEQ ID NO:1 in which at least one but no more than twenty amino acids are deleted from the N-terminal side.

43. The pharmaceutical composition of claim 41, wherein said truncated peptide comprises the amino acid sequence set forth in SEQ ID NO:1 in which at least one but no more than fifteen amino acids are deleted from the N-terminal side.

44. The pharmaceutical composition of claim 41, wherein said truncated peptide comprises the amino acid sequence set forth in SEQ ID NO:1 in which at least one but no more than ten amino acids are deleted from the N-terminal side.

45. The pharmaceutical composition of claim 41, wherein said truncated peptide comprises the amino acid sequence set forth in SEQ ID NO:1 in which at least one but no more than five amino acids are deleted from the N-terminal side.

46. The pharmaceutical composition of claim 41, wherein said truncated peptide has the sequence shown in SEQ ID NO:2.

47. The pharmaceutical composition of claim 41, wherein said truncated peptide further comprises a mutation, wherein said mutation is selected from the group consisting of a deletion, a substitution, and an insertion.

48. The pharmaceutical composition of claim 40, wherein said compound is formulated for treatment of stomach cancer, colorectal cancer, lung cancer, ovarian cancer, liver cancer, or pancreatic cancer.

49. A method of treating cancer in a subject in need of such treatment, comprising administering the pharmaceutical composition of claim 40.

50. The method of claim 49, wherein said cancer is stomach cancer, colorectal cancer, lung cancer, ovarian cancer, liver cancer, or pancreatic cancer.

51. The method of claim 49, wherein said pharmaceutical composition is administered intramuscularly or intratumorally.

52. A method of inhibiting growth of a cancerous tumor and reducing the present of blood vessels in said tumor, comprising administering the pharmaceutical composition of claim 40.

53. A method for treating cardiovascular and renal disease, comprising administering a peptide having the sequence shown in SEQ ID NO:1, wherein said peptide induces or generates macroangiogenesis.


SEQUENCE LISTING (14 sequences)

SEQ ID NO: 1 - protein, 52 aa, Homo sapiens
YRQSMNNFQGLRSFGCRFGTCTVQKLAHQIYQFTDKDKDNVAPRSKISPQGY

SEQ ID NO: 2 - protein, 31 aa, Artificial
  An artificially synthesized peptide sequence
TVQKLAHQIYQFTDKDKDNVAPRSKISPQGY

SEQ ID NO: 3 - DNA, 19 bp, Artificial
  An artificially synthesized primer sequence
gctcctcctgagcgcaagt

SEQ ID NO: 4 - DNA, 23 bp, Artificial
  An artificially synthesized primer sequence
tcgtcatactcctgcttgctgat

SEQ ID NO: 5 - protein, 100 aa, Artificial
  An artificially synthesized peptide sequence
MKLVSVALMYLGSLAFLGADTRSSVQKLAHQIYQFTDKDKDNVAPRSKISPQGYGRRRRRSLPEAGPGRTLVSSKPQAHGAPAPPSGSAPHFLDYKDHDG

SEQ ID NO: 6 - protein, 172 aa, Artificial
  An artificially synthesized peptide sequence
MKLVSVALMYLGSLAFLGADTARLDVASEFRKKWNKWALSRGKRELRMSSSYPTGLADVKAGPAQTLIRPQDMKGASRSPEDSSPDAARIRVKRSSVQKLAHQIYQFTDKDKDNVAPRSKISPQGYGRRRRRSLPEAGPGRTLVSSKPQAHGAPAPPSGSAPHFLDYKDHDG

SEQ ID NO: 7 - protein, 78 aa, Artificial
  An artificially synthesized peptide sequence
MKLVSVALMYLGSLAFLGADTRSTVQKLAHQIYQFTDKDKDNVAPRSKISPQGYGSDYKDHDGDYKDHDIDYKDDDDK

SEQ ID NO: 8 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 9 - DNA, 29 bp, Artificial
  Antisense siRNA Oligonucleotide Template
aagctggcacaccagatctaccctgtctc

SEQ ID NO: 10 - DNA, 29 bp, Artificial
  Sense siRNA Oligonucleotide Template
aagtagatctggtgtgccagccctgtctc

SEQ ID NO: 11 - DNA, 29 bp, Artificial
  Antisense siRNA Oligonucleotide Template
aagctggtttccgtcgccctgcctgtctc

SEQ ID NO: 12 - DNA, 29 bp, Artificial
  Sense siRNA Oligonucleotide Template
aacagggcgacggaaaccagccctgtctc

SEQ ID NO: 13 - DNA, 29 bp, Artificial
  Antisense siRNA Oligonucleotide Template
aagcgctaccgccagagcatgcctgtctc

SEQ ID NO: 14 - DNA, 29 bp, Artificial
  Sense siRNA Oligonucleotide Template
aacatgctctggcggtagcgccctgtctc